(19) United States
(12) Patent Application Publication
(10) Pub. No.: US 2022/0036992 A1
(43) Pub. Date: Feb. 3, 2022

(54) AUTOMATIC DEVICE CONFIGURATION VIA A NETWORK SERVICE

(71) Applicant: MEDTRONIC MINIMED, INC., Northridge, CA (US)

(72) Inventors: Samuel Finney, Los Angeles, CA (US); Ben Wu, Pasadena, CA (US); Afshin Bazargan, Simi Valley, CA (US); Mehmet Tumer, Studio City, CA (US)

(21) Appl. No.: 16/944,067

(22) Filed: Jul. 30, 2020

(51) Int. Cl. G16H 20/17; G16H 10/60; G16H 40/67; G06F 8/71; A61M 5/172

(52) U.S. Cl. CPC G16H 20/17; G16H 10/60; A61M 5/1723; G06F 8/71; G16H 40/67

(57) ABSTRACT

Methods and systems are provided for automatically configuring a first medical device. In accordance with the methods and the systems, a computing system can obtain prescription information for a patient. The prescription information comprises a device prescription. The computing system can transform the prescription information into first configuration data comprising therapy settings for a first medical device referenced by the device prescription. The computing system can cause automatic configuration of the first medical device based on communicating the first configuration data to the first medical device.

### TECHNICAL FIELD

The present technology is generally related to device autoconfiguration and, more specifically, to automatic device configuration via a network service.

### BACKGROUND

Portable computing devices (e.g., smartphones, laptops, and tablets) are used to control other devices in an increasing number of fields. For example, in the medical device field, there is a growing trend towards controlling personal medical devices, such as insulin infusion devices that are worn by a patient, using portable computing devices. Portable computing devices can even be used to control and communicate with on-body medical devices, such as patch pumps and sensors. Using such non-medical devices as part of a medical device system can provide a number of advantages.

The advantages include eliminating the need for a dedicated control device. This can make the cost of ownership lower. In addition, patients can be discreet in managing their disease and use a familiar user interface which shortens the learning curve. This can also provide connectivity to server systems (e.g., cloud-based systems) that are used to monitor patients and control delivery of medication to a patient. Non-medical smart devices can upload data for analysis by health care providers (HCPs) and AI-based systems. Non-medical smart devices can also receive regular updates to therapy management software. For instance, these updates can update medical control application software (or key parameters thereof) to customize and improve therapy. Connectivity also provides the means to alert caregivers and HCPs in emergency situations when concerning trends are present. A medical control application can also use health related data from the non-medical smart device and other apps/systems to improve therapy for the patient.

A number of insulin pump systems are designed to deliver accurate and measured doses of insulin via infusion sets (e.g., an infusion set that delivers the insulin through a small diameter tube that terminates at a cannula inserted under the patient's skin). In lieu of a syringe, the patient can simply activate the insulin pump to administer an insulin bolus as needed, for example, in response to the patient's current blood glucose (BG) level. A patient can measure his BG level using a BG measurement device, such as a test strip meter, a continuous glucose measurement system, or the like. BG measurement devices use various methods to measure the BG level of a patient, such as a sample of the patient's blood, a sensor in contact with a bodily fluid, an optical sensor, an enzymatic sensor, or a fluorescent sensor. When the BG measurement device has generated a BG measurement, the measurement is typically displayed on the BG measurement device. A continuous glucose monitoring system can monitor the patient's sensor glucose (SG) level (e.g., subcutaneous tissue glucose level) in real-time. This allows insulin delivery dosages to be calculated in real-time by a software algorithm (e.g., a closed-loop algorithm) based on measured sensor glucose level.

The insulin pump, continuous glucose monitoring (CGM) device, and other devices, such as a smartphone and a Blood Glucose Monitor (BGM), can be different parts of an insulin infusion system. The various devices that are part of the insulin infusion system can form a wireless body area network that can be used, for example, to exchange monitor and therapy (control) data among multiple medical devices that are either worn on or near a patient's body. For instance, measured glucose values (SG values) can be transferred wirelessly among devices within the body area network. Insulin pumps and CGM devices may also be configured to communicate with remote control devices, monitoring or display devices, BG meters, and other devices associated with such an infusion system. For example, a CGM sensor may include or be coupled to a wireless radio frequency (“RF”) transmitter that communicates with other devices that are part of an infusion system such as a handheld remote control (also called a command control device (CCD)) that communicates with the infusion pump device using wireless communication technologies such as classical Bluetooth® (BT) or Bluetooth Low Energy® (BLE) technologies.

Complex medical devices, such as insulin pumps, often require configuration prior to use. The process of configuring a system for insulin infusion therapy takes time and a particular level of expertise and knowledge. Many patients do not have the expertise or knowledge that is needed to properly configure their systems since this configuration may rely on patient-specific system parameters (e.g., therapy settings, control algorithm settings, or other parameters). In most cases a physician or other healthcare provider helps each patient configure their devices with patient-specific system parameters that are unique to each patient. Therapy settings, control algorithm settings, or other parameters may affect operation of the medical device for therapy purposes, and may also alter device behaviors such as operating modes, treatment methods, or safety restrictions. The process of configuring medical devices and keeping patient-specific system parameters up to date can be time-consuming and burdensome.

Accordingly, it is desirable to reduce the time and effort involved in configuring devices (e.g., insulin pumps, glucose sensors, and other medical devices) and to enable simplified configuration of medical devices. For example, it would be desirable to simplify the process of setting up patient-specific therapy settings and parameters for such devices. Furthermore, other desirable features and characteristics will become apparent from the subsequent detailed description and the appended claims, taken in conjunction with the accompanying drawings and the foregoing technical field and background.

### BRIEF SUMMARY

Methods and systems are provided for automatically configuring a first medical device. In accordance with the methods and the systems, a computing system can obtain prescription information for a patient. The prescription information comprises a device prescription. The computing system can transform the prescription information into first configuration data comprising therapy settings for a first medical device referenced by the device prescription. The computing system can cause automatic configuration of the first medical device based on communicating the first configuration data to the first medical device. In some embodiments, the first medical device comprises at least one of a group including an insulin infusion device and a glucose sensor.

In some embodiments, the device prescription comprises identifying information for the patient, a model of the first medical device, and a therapy type prescribed for the patient. In some embodiments, the prescription information comprises one or more of: basic patient-specific data for the patient, the basic patient-specific data comprising: physical, metabolic or anatomical data about the patient, and information about medical conditions; patient lifestyle data for the patient, the patient lifestyle data comprising: data regarding a diet of the patient, and data regarding exercise characteristics of the patient; information about a medical condition being targeted for therapy and information about medical conditions other than the condition being targeted for therapy; information regarding therapy history of the patient; and known therapy settings from prior medical devices of the patient.

In some embodiments, the prescription information can be transformed into the first configuration data by selecting an appropriate therapy configuration for the first medical device based on the prescription information, and determining therapy parameters and settings for the first medical device based on the prescription information. In some embodiments, the appropriate therapy configuration comprises: one or more therapy algorithms to be executed by the first medical device for the patient; and one or more operating modes of the first medical device to be used for the patient.

In some embodiments, the therapy parameters and settings comprise appropriate device configuration parameters or settings for configuring the first medical device of the patient, and therapy configuration parameters for configuring the first medical device of the patient. In some embodiments, the computing system can combine the appropriate therapy configuration and the therapy parameters and settings for the first medical device into the first configuration data, and maintain the first configuration data in a database. In some embodiments, the first configuration data is structured as at least one software image.

In some embodiments, the computing system can transform the prescription information into second configuration data comprising therapy settings for a second medical device referenced by the prescription information, where the first medical device is a different model from the second medical device.

Methods and systems are provided for configuring therapy settings of a medical device for a specific patient. Patient data input via a non-medical device can be received. A value of a total daily dose of insulin for the specific patient can be obtained from the patient data. Based upon the value of the total daily dose, a set of therapy settings for the specific patient can be computed. The medical device can be automatically programmed with the set of therapy settings to complete set up of the medical device so that the medical device is configured for the specific patient. The set of therapy settings for the specific patient can include, for example, a basal rate for the specific patient, an insulin sensitivity factor for the specific patient, an insulin to carbohydrate ratio for the specific patient, an active insulin time for the specific patient, a maximum bolus limit for the specific patient, a maximum basal rate for the specific patient, a bolus increment for the specific patient, and a basal increment for the specific patient. Depending on the implementation, the set of therapy settings for the specific patient can be computed by one of the non-medical device, the medical device, and a server system that hosts a cloud-based service.

In some embodiments, the patient data comprises a weight of the specific patient, and the value of the total daily dose of insulin for the specific patient can be obtained from the patient data by computing the value of the total daily dose based on the weight of the specific patient. Depending on the implementation, the value of the total daily dose can be computed by one of the non-medical device, the medical device, and a server system that hosts a cloud-based service.

In some embodiments, the patient data comprises the value of the total daily dose of insulin for the specific patient and the value of the total daily dose of insulin for the specific patient can be obtained from the patient data by extracting the value of the total daily dose of insulin for the specific patient from the patient data

In some embodiments, the non-medical device can confirm whether a value of the total daily dose is correct. When the value of the total daily dose is incorrect an updated value of the total daily dose can be received via the non-medical device, and the set of therapy settings for the specific patient can be computed based upon the updated value of the total daily dose.

A computing system is provided including at least one processor device; and a non-transitory processor-readable medium operatively associated with the at least one processor device, the processor-readable medium comprising executable instructions configurable to cause the at least one processor device to perform a method. In accordance with the method, patient data input via a non-medical device can be received, and from the patient data, a value of a total daily dose of insulin for a specific patient can be obtained. A set of therapy settings for the specific patient can be computed based upon the value of the total daily dose, and a medical device can be automatically programed with the set of therapy settings to complete set up of the medical device so that the medical device is configured for the specific patient.

An electronic device is provided including at least one processor device; and a non-transitory processor-readable medium operatively associated with the at least one processor device, the processor-readable medium comprising executable instructions configurable to cause the at least one processor device to perform a method. In accordance with the method, patient data input via a non-medical device can be received, and from the patient data, a value of a total daily dose of insulin for a specific patient can be obtained. Based upon the value of the total daily dose, a set of therapy settings can be computed for automatically programming a medical device of the specific patient so that the medical device is configured for the specific patient, and the set of therapy settings can be transmitted to the medical device of the specific patient.

An electronic device is provided including at least one processor device; and a non-transitory processor-readable medium operatively associated with the at least one processor device, the processor-readable medium comprising executable instructions configurable to cause the at least one processor device to perform a method. In accordance with the method, patient data input via a non-medical device can be received, and from the patient data, a value of a total daily dose of insulin for a specific patient can be obtained. Based upon the value of the total daily dose, a set of therapy settings for the specific patient can be computed. The processor device can be automatically programmed with the set of therapy settings for the specific patient so that the medical device is configured for the specific patient. In some embodiments, the medical device comprises an insulin infusion device, and the set of therapy settings for the specific patient comprise: a basal rate for the specific patient, an insulin sensitivity factor for the specific patient, an insulin to carbohydrate ratio for the specific patient, an active insulin time for the specific patient, a maximum bolus limit for the specific patient, a maximum basal rate for the specific patient, a bolus increment for the specific patient, and a basal increment for the specific patient.

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

### BRIEF DESCRIPTION OF THE DRAWINGS

A more complete understanding of the subject matter may be derived by referring to the detailed description and claims when considered in conjunction with the following figures, wherein like reference numbers refer to similar elements throughout the figures.

FIG. 1 is a schematic diagram of an infusion system;

FIG. 2 is a simplified block diagram representation of an example embodiment of a communication system;

FIG. 3 is a simplified block diagram representation of a computer-based or processor-based device in accordance with the disclosed embodiments;

FIG. 4 is a block diagram of a patient monitoring system in accordance with the disclosed embodiments;

FIG. 5 is a flow diagram that illustrates an automatic medical device configuration method for configuring one or more medical devices that will be provided to a patient in accordance with the disclosed embodiments;

FIG. 6 is a flow diagram that illustrates a settings generation method for generating settings with which to configure one or more medical devices that will be provided to a patient in accordance with the disclosed embodiments;

FIG. 7 is a flowchart that illustrates an automatic medical device configuration method for configuring one or more medical devices that will be provided to a patient in accordance with the disclosed embodiments;

FIG. 8 is a flowchart that illustrates a method for configuring therapy settings of a medical device for a specific patient during startup mode of the medical device in accordance with the disclosed embodiments;

FIG. 9 is a flow diagram that illustrates a method for configuring therapy settings of a medical device for a specific patient during startup mode of the medical device in accordance with the disclosed embodiments;

FIG. 10 is a flow diagram that illustrates another method for configuring therapy settings of a medical device for a specific patient during startup mode of the medical device in accordance with the disclosed embodiments; and

FIG. 11 is a flow diagram that illustrates another method for configuring therapy settings of a medical device for a specific patient during startup mode of the medical device in accordance with the disclosed embodiments.

### DETAILED DESCRIPTION

The following detailed description is merely illustrative in nature and is not intended to limit the embodiments of the subject matter or the application and uses of such embodiments. As used herein, the word “exemplary” means “serving as an example, instance, or illustration.” Any implementation described herein as exemplary is not necessarily to be construed as preferred or advantageous over other implementations. Furthermore, there is no intention to be bound by any expressed or implied theory presented in the preceding technical field, background, brief summary or the following detailed description.

Techniques and technologies may be described herein in terms of functional and/or logical block components, and with reference to symbolic representations of operations, processing tasks, and functions that may be performed by various computing components or devices. Such operations, tasks, and functions are sometimes referred to as being computer-executed, computerized, software-implemented, or computer-implemented. It should be appreciated that the various block components shown in the figures may be realized by any number of hardware, software, and/or firmware components configured to perform the specified functions. For example, an embodiment of a system or a component may employ various integrated circuit components, e.g., memory elements, digital signal processing elements, logic elements, look-up tables, or the like, which may carry out a variety of functions under the control of one or more microprocessors or other control devices.

When implemented in software, firmware, or processor-readable instructions, various elements of the systems described herein are essentially the code segments or instructions that perform the various tasks. In certain embodiments, the program or code segments are stored in a tangible processor-readable medium, which may include any medium that can store or transfer information. Examples of a non-transitory and processor-readable medium include an electronic circuit, a semiconductor memory device, a ROM, a flash memory, an erasable ROM (EROM), a floppy diskette, a CD-ROM, an optical disk, a hard disk, or the like.

Exemplary embodiments of the subject matter are described herein in terms of patients and medical devices, such as portable electronic medical devices. However, it should be appreciated that the techniques disclosed herein are equally applicable to users and devices in non-medical contexts. Although many different applications are possible, the following description focuses on embodiments that incorporate an insulin infusion device (or insulin pump) as part of an infusion system deployment. For the sake of brevity, conventional techniques related to infusion system operation, insulin pump and/or infusion set operation, and other functional aspects of the systems (and the individual operating components of the systems) may not be described in detail here. Examples of infusion pumps may be of the type described in, but not limited to, U.S. Pat. Nos. 4,562,751; 4,685,903; 5,080,653; 5,505,709; 5,097,122; 6,485,465; 6,554,798; 6,558,320; 6,558,351; 6,641,533; 6,659,980; 6,752,787; 6,817,990; 6,932,584; and 7,621,893; each of which are herein incorporated by reference.

Generally, a fluid infusion device includes a motor or other actuation arrangement that is operable to linearly displace a plunger (or stopper) of a fluid reservoir provided within the fluid infusion device to deliver a dosage of fluid, such as insulin, to the body of a user. Dosage commands that govern operation of the motor may be generated in an automated manner in accordance with the delivery control scheme associated with a particular operating mode, and the dosage commands may be generated in a manner that is influenced by a current (or most recent) measurement of a physiological condition in the body of the user. For example, in a closed-loop or automatic operating mode, dosage commands may be generated based on a difference between a current (or most recent) measurement of the interstitial fluid glucose level in the body of the user and a target (or reference) glucose setpoint value. In this regard, the rate of infusion may vary as the difference between a current measurement value and the target measurement value fluctuates. For purposes of explanation, the subject matter is described herein in the context of the infused fluid being insulin for regulating a glucose level of a user (or patient); however, it should be appreciated that many other fluids may be administered through infusion, and the subject matter described herein is not necessarily limited to use with insulin.

From the perspective of health care providers and end users, the process of configuring medical devices such as insulin pumps can be time-consuming and burdensome. A doctor typically prescribes a particular type of medical device that would be the best model for a particular patient given the patient's particular characteristics and manually configures different parameters of that particular type of medical device in accordance with a therapy regimen that the doctor deems suitable to address the patient's needs. For example, in some cases, a physician can select a particular model of a medical device that can provide appropriate therapy for a particular patient (e.g., a device having certain feature sets or capabilities for therapy/treatment). The medical device can then be configured or programmed with configuration parameters by a user, such as the patient or a health care provider, by entering configuration parameters into the medical device. For example, in many cases, a physician can configure or program a particular medical device by calculating and manually entering configuration parameters that are determined based on the therapy settings/needs of the particular patient (such as his/her treatment history, insulin dosing, insulin sensitivity, etc.). For instance, in the case of an insulin pump, is often necessary for a doctor or other healthcare provider to manually configure certain parameters and settings of the insulin pump. This configuration is typically different for each patient, because it considers each patient's individual characteristics. The therapy regimen that is prescribed for each patient can vary dramatically from patient to patient. Each patient may not only have his/her own therapy regimen, but that therapy regimen can also vary over time depending on a number of variables. As such, the configuration of medical devices can be a very manual and time-consuming process.

Some devices may require more frequent or more involved configuration for a variety of reasons. For example, some devices may be at least partially disposable (e.g., a patch-type insulin pump that is delivered to an end user in large volumes, used and then replaced after a period of use). As another example, a particular patient may have an insulin pump that includes a disposable part and a durable part, where the particular patient may use the durable part for an extended period of time, but the disposable part can have a limited life and be used for a relatively short period of time, after which it is discarded and replaced with a new disposable part that can be used in conjunction with the existing durable part. In some cases, medical devices or parts thereof (e.g., the durable parts) may be delivered to an end user in large volumes. In such cases, a configuration process is typically repeated for each of the medical devices or parts (e.g., each device may be separately configured for that particular patient to take into account the particular patient's needs).

In addition, multiple devices may be issued to a patient to support maintenance without interruption in therapy (e.g., charging one device while another is in use.). It is not unusual for a particular patient to have multiple, ambulatory medical devices that he/she uses to provide insulin therapy. For example, a particular patient may have several insulin pumps that he/she utilizes on a rotating basis (e.g., switched back and forth for maintenance, charging, etc.).

Currently, some device manufacturers make and sell several different medical device models (e.g., insulin pump models) to provide different therapies to patients. Although the device hardware may be common between the different medical device models, the different medical device models may use different software and may be inventoried and shipped using different part numbers. device manufacturers may provide devices to patients that require hardware and/or software configuration for each particular patient prior to being used. In this case, a configuration process needs to be performed by the prescribing physician, and as noted above, the configuration of medical devices can be a very manual and time-consuming process. This may include, for example, selection of high-level therapy features, such as closed-loop algorithm types, as well as low-level therapy parameters. This configuration process may also take into account the medical needs of the patient, the physician's assessment of the patient's ability to manage different therapy features, and cost considerations (if therapy options are priced at different levels.)

Future medical devices may support a broader variety of configurable therapy parameters or selectable therapy algorithms, requiring even more configuration effort to adjust each device to a patient. Future technological developments may make current methods of device configuration too difficult to be feasible. The number of configurable therapy parameters may grow as therapy algorithms become more sophisticated, increasing the burden of manual entry as well as the risk of errors. Future devices may also have more limited on-board user interfaces, or none at all, as automation increases and the need for manual patient control diminishes.

To address these issues, disclosed herein are techniques related to automatic device configuration via a network service. In some embodiments, such techniques may be practiced in the context of a medical device system, such as an insulin infusion system. The medical device system can include a cloud-based server system for physician entry of configuration parameters along with partial automation or guidance in the selection of these values. This entry process can be completed, for example, when a patient is first prescribed a device. The cloud-based system can automatically feed configuration information to a patient's medical devices when they are received by the patient and placed into service. This process reduces physician workload, improves the patient experience, and decreases the risk of errors during repetitive entry of configuration data.

Thus, instead of configuring a particular medical device by calculating and manually entering configuration parameters into a particular medical device for a particular patient, the disclosed embodiments can allow for at least some of the configuration to be automated (as opposed to applying rules of thumb, or computing values using equations or charts, etc.). In some embodiments, a cloud-based system allows a user (e.g., the physician or other health care provider) to simply input a set of data for the particular patient, and the system then automatically generates and delivers configuration parameters to a particular medical device that can be used to configure that particular medical device for that particular patient (and possibly other medical devices for that particular patient).

This allows for the medical devices to be delivered to the patient in an unconfigured state, and then configured “in the field” without requiring manual configuration or the assistance of a healthcare provider. This simplifies the process of programming or configuring the devices and reduces the need for intervention by a health care provider in the configuration process. Automating the initial configuration of device parameters not only reduces physician workload, but also improves the patient experience, and decreases the risk of errors during repetitive entry of configuration data. Automation may also lead to an improved patient experience during the initial stages of treatment because a cloud-based solution for automatically determining therapy parameters may yield a more optimal device configuration than manual methods. In addition to allowing for initial configuration, this cloud-based system also allows for multiple medical devices to be updated without requiring manual configuration or the assistance of a healthcare provider.

The benefits are even more pronounced for future devices requiring more frequent configuration (e.g., either because a patient is issued multiple devices or because devices have a shorter service life). For example, multiple medical devices for a particular patient can be configured with appropriate configuration parameters (e.g., configuration data and appropriate settings) for that particular patient using a cloud-based system so that they operate consistently and are synchronized in terms of their operating characteristics. In other words, the device configuration parameters can be automatically sent to different medical devices of a particular patient and used to configure the different medical devices thereby eliminating the need for the patient or a healthcare provider of the patient to manually configure many different medical devices for that particular patient.

Another advantage of the disclosed embodiments is that generic, commercial-off-the-shelf (COTS) medical devices can be delivered to many different patients, and then customized for each particular patient so that they are configured with appropriate configuration data and appropriate settings for that particular patient. For instance, generic medical devices that are in an unprogrammed/unconfigured state (e.g., that have a blank configuration payload and that has not been configured for the particular patient) can be shipped to many different patients. Each generic medical devices can then be programmed with the correct therapy model and configuration parameters so that the generic medical device becomes customized for a particular patient. In some implementations, when a particular customer receives one of these generic pumps, they can log into the cloud-based computing system and download a prescribed/appropriate therapy model to customize that generic pump so that it operates according to a specific therapy model (i.e., operates as a specific pump model). In addition, all of the configuration parameters can also be downloaded so that the pump is configured to with appropriate configuration parameters (configuration data and appropriate settings) for that particular patient. The complete configuration bundle can be transmitted to the medical device once it is received by the patient. The configuration bundle may be structured in a number of different ways: a single common software image, with configurable therapy algorithms and parameters; one software image per therapy algorithm with configurable parameters; or unique software images for each patient.

The disclosed embodiments can also simplify inventory management for medical device manufacturers and distributors by reducing the number of different models of medical devices that need to be produced for different patients because operation of a generic medical device can be customized for many different patients. The generic medical device can be sent to customers, such as patients, and then configured with the appropriate therapy model thus becoming a patient-specific medical device. As such, instead of stocking a number of different pump models (e.g., 12 different pump models), a single configurable pump can be shipped to different customers. This process may enable a more streamlined approach to providing different types of therapy. Moreover, the patient does not need to worry about ordering a specific model. Instead, he/she can order a generic model and configure it, for example, by logging into cloud-based computing system (e.g., CARELINK®) using, for example, their smartphone, and the cloud-based computing system delivers all configuration information needed to configure the device in accordance with a specific therapy model. This configuration process allows physicians to have the necessary control of patient device settings without unduly restricting future device design possibilities. This not only simplifies things for the end user and health care providers, but also simplifies supply chain management by reducing stocking, inventory, turn-around times, etc.

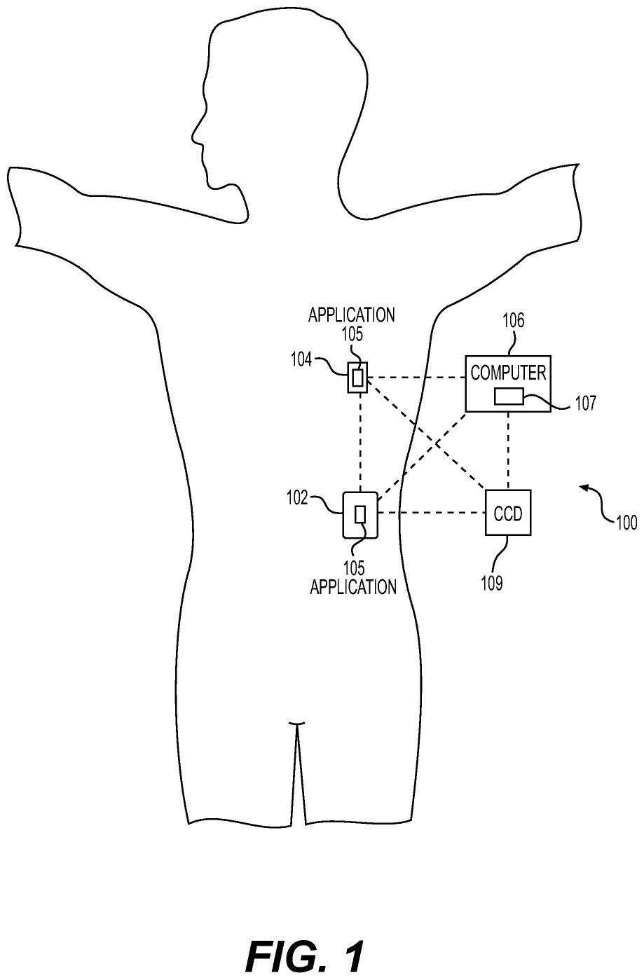

Turning now to FIG. 1, an infusion system 100 includes, without limitation, a fluid infusion device (e.g., an infusion pump) 102, a sensing arrangement 104, a command control device (CCD) 109, and a computer 106. The components of an infusion system 100 may be realized using different platforms, designs, and configurations, and the embodiment shown in FIG. 1 is not exhaustive or limiting. In practice, the infusion device 102 and the sensing arrangement 104 are secured at desired locations on the body of a user (e.g., a patient), as illustrated in FIG. 1. In this regard, the locations at which the infusion device 102 and the sensing arrangement 104 are secured to the body of the user in FIG. 1 are provided only as a representative, non-limiting, example. The elements of the infusion system 100 may be similar to those described in United States Patent No. 8,674,288, the subject matter of which is hereby incorporated by reference in its entirety.

In the illustrated embodiment of FIG. 1, the infusion device 102 is designed as a portable medical device suitable for infusing a fluid, a liquid, a gel, and/or medicament into the body of a user. In exemplary embodiments, the infused fluid is insulin, although many other fluids may be administered through infusion such as, but not limited to, HIV drugs, drugs to treat pulmonary hypertension, iron chelation drugs, pain medications, anti-cancer treatments, medications, vitamins, hormones, or the like. In some embodiments, the fluid may include a nutritional supplement, a dye, a tracing medium, a saline medium, a hydration medium, or the like.

The sensing arrangement 104 generally represents the components of the infusion system 100 configured to sense, detect, measure or otherwise quantify a condition of the user, and may include a sensor, a monitor, or the like, for providing data indicative of the condition that is sensed, detected, measured or otherwise monitored by the sensing arrangement. In this regard, the sensing arrangement 104 may include electronics and enzymes reactive to a biological condition, such as a blood glucose level, or the like, of the user, and provide data indicative of the blood glucose level to the infusion device 102, the CCD 109 and/or the computer 106. For example, the infusion device 102, the CCD 109 and/or the computer 106 may include a display for presenting information or data to the user based on the sensor data received from the sensing arrangement 104, such as, for example, a current glucose level of the user, a graph or chart of the user's glucose level versus time, device status indicators, alert messages, or the like. In other embodiments, the infusion device 102, the CCD 109 and/or the computer 106 may include electronics and software that are configured to analyze sensor data and operate the infusion device 102 to deliver fluid to the body of the user based on the sensor data and/or preprogrammed delivery routines. Thus, in exemplary embodiments, one or more of the infusion device 102, the sensing arrangement 104, the CCD 109, and/or the computer 106 includes a transmitter, a receiver, and/or other transceiver electronics that allow for communication with other components of the infusion system 100, so that the sensing arrangement 104 may transmit sensor data or monitor data to one or more of the infusion device 102, the CCD 109 and/or the computer 106.

Still referring to FIG. 1, in various embodiments, the sensing arrangement 104 may be secured to the body of the user or embedded in the body of the user at a location that is remote from the location at which the infusion device 102 is secured to the body of the user. In various other embodiments, the sensing arrangement 104 may be incorporated within the infusion device 102. In some embodiments, the sensing arrangement 104 may be separate and apart from the infusion device 102, and may be, for example, part of the CCD 109. In such embodiments, the sensing arrangement 104 may be configured to receive a biological sample, analyte, or the like, to measure a condition of the user.

In some embodiments, the CCD 109 and/or the computer 106 may include electronics and other components configured to perform processing, delivery routine storage, and to control the infusion device 102 in a manner that is influenced by sensor data measured by and/or received from the sensing arrangement 104. By including control functions in the CCD 109 and/or the computer 106, the infusion device 102 may be made with more simplified electronics. However, in other embodiments, the infusion device 102 may include all control functions, and may operate without the CCD 109 and/or the computer 106. In various embodiments, the CCD 109 may be a portable electronic device. In addition, in various embodiments, the infusion device 102 and/or the sensing arrangement 104 may be configured to transmit data to the CCD 109 and/or the computer 106 for display or processing of the data by the CCD 109 and/or the computer 106.

In some embodiments, the CCD 109 and/or the computer 106 may provide information to the user that facilitates the user's subsequent use of the infusion device 102. For example, the CCD 109 may provide information to the user to allow the user to determine the rate or dose of medication to be administered into the user's body. In some embodiments, the CCD 109 may provide information to the infusion device 102 to autonomously control the rate or dose of medication administered into the body of the user. In some embodiments, the sensing arrangement 104 may be integrated into the CCD 109. Such embodiments may allow the user to monitor a condition by providing, for example, a sample of his or her blood to the sensing arrangement 104 to assess his or her condition. In some embodiments, the sensing arrangement 104 and the CCD 109 may be used for determining glucose levels in the blood and/or body fluids of the user without the use of, or necessity of, a wire or cable connection between the infusion device 102 and the sensing arrangement 104 and/or the CCD 109.

In some embodiments, the sensing arrangement 104 and/or the infusion device 102 are cooperatively configured to utilize a closed-loop system for delivering fluid to the user. Examples of sensing devices and/or infusion pumps utilizing closed-loop systems may be found at, but are not limited to, the following U.S. Pat. Nos. 6,088,608, 6,119,028, 6,589,229, 6,740,072, 6,827,702, 7,323,142, and 7,402,153 or United States Patent Application Publication No. 2014/0066889, all of which are incorporated herein by reference in their entirety. In such embodiments, the sensing arrangement 104 is configured to sense or measure a condition of the user, such as, blood glucose level or the like. The infusion device 102 is configured to deliver fluid in response to the condition sensed by the sensing arrangement 104. The sensing arrangement 104 continues to sense or otherwise quantify a current condition of the user, thereby allowing the infusion device 102 to deliver fluid continuously in response to the condition currently (or most recently) sensed by the sensing arrangement 104. In some embodiments, the sensing arrangement 104 and/or the infusion device 102 may be configured to utilize the closed-loop system only for a portion of the day, for example only when the user is asleep or awake.

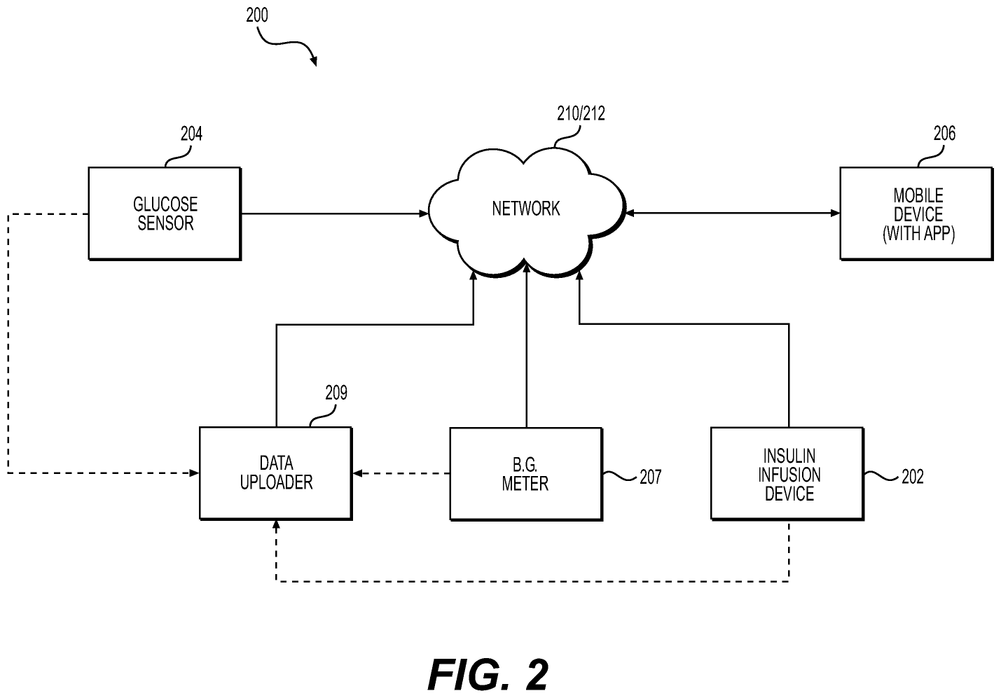

FIG. 2 is a simplified block diagram representation of an exemplary embodiment of a communication system 200 that is suitably configured to support the techniques and methodologies described in more detail below. The system 200 supports users of insulin infusion devices and performs various techniques and methods to help users (patients, caregivers, healthcare providers, parents, etc.) manage the use of insulin infusion devices. It should be appreciated that FIG. 2 depicts one possible implementation of a communication system, and that other arrangements, architectures, and deployments can be provided if so desired. The system 200 (which has been simplified for purposes of illustration) generally includes or cooperates with the following components, without limitation: a mobile device 206; an insulin infusion device 202; a blood glucose meter 207; a continuous glucose sensor 204; and an optional data uploader 209. The mobile device 206 (which can perform the role of a client device in some embodiments) is owned or operated by the user, i.e., a diabetic patient. The insulin infusion device 202, the blood glucose meter 207, and the glucose sensor 204 are components of an insulin infusion system that is used by the patient to treat diabetes. The system 200 may also include or cooperate with the optional data uploader component 209.

The various components of the system 200 can be used to collect and analyze input data for the patient that can originate from various sources, including an insulin infusion device, a glucose sensor or meter, a mobile device operated by a user of the insulin infusion device, or other components or computing devices that are compatible with the system, such as a data uploader. These and other alternative arrangements are contemplated by this disclosure. To this end, some embodiments of the system may include additional devices and components that serve as data sources, data processing units, etc. For example, the system may include any or all of the following elements, without limitation: computer devices or systems; patient monitors; healthcare provider systems; data communication devices; and the like. It should be appreciated that the insulin infusion device 202 can be an optional component in some applications (for example, for Type 2 diabetes patients). For such applications, another diabetes management device and/or the mobile device 206 can function in an equivalent manner to support the system 200.

At a minimum, the mobile device 206 is communicatively coupled to a network 210. In certain embodiments, the insulin infusion device 202, the blood glucose meter 207, and/or the continuous glucose sensor 204 are also communicatively coupled to the network 210 to facilitate the uploading of relevant data to a remote server system (not illustrated). Alternatively, or additionally, the insulin infusion device 202, the blood glucose meter 207, and the continuous glucose sensor 204 provide relevant data to the data uploader component 209, which in turn uploads the data to other systems (not illustrated) via the network 210.

FIG. 2 depicts the network 210 in a simplified manner. In practice, the system 200 may cooperate with and leverage any number of wireless and any number of wired data communication networks maintained or operated by various entities and providers. Accordingly, communication between the various components of the system 200 may involve multiple network links and different data communication protocols. In this regard, the network 210 can include or cooperate with any of the following, without limitation: a local area network; a wide area network; the Internet; a personal area network; a cellular communication network; a satellite communication network; a video services or television broadcasting network; a network onboard a vehicle; or the like. In addition, the various components can also communicate directly with each other using NFMI radio communications; NFeMI radio communications, BLE® communications, classical Bluetooth® (BT) communications, WLAN (or “Wi-Fi”) communications, or indirectly with each other using WLAN or cellular communications, as will be described below. The components of the system may be suitably configured to support a variety of wireless and wired data communication protocols, technologies, and techniques as needed for compatibility with the network 210.

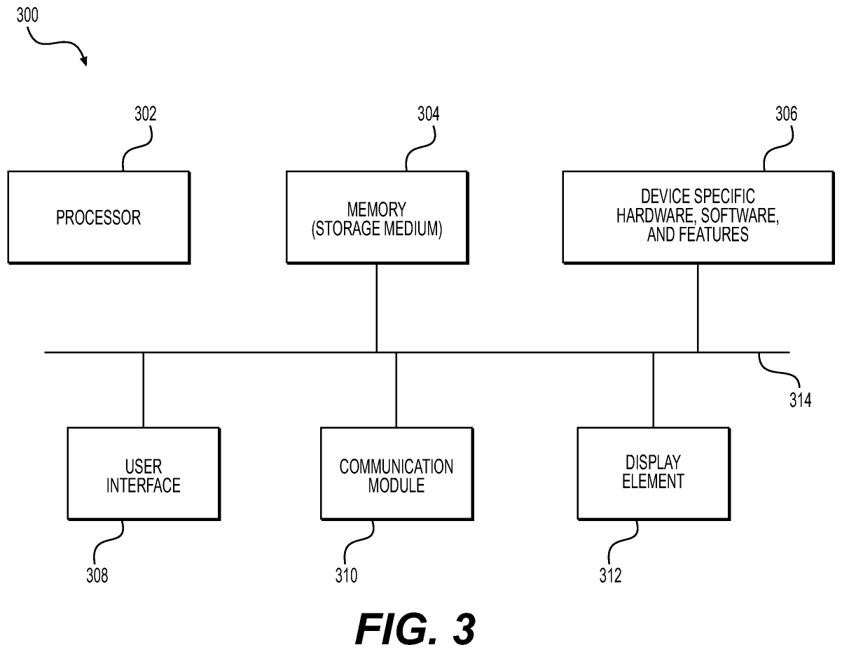

The mobile device 206 can be realized using a variety of different device platforms. For example, the mobile device 206 can be implemented as any of the following, without limitation: a cellular telephone or smartphone; a portable computer (e.g., a laptop, a tablet, or a netbook computer); a portable media player; a portable video game device; a portable medical device; a navigation device such as a global positioning system (GPS) device; a wearable computing device; an electronic toy or game; etc. In accordance with certain exemplary embodiments, the mobile device 206 supported by the system 200 is implemented as a computer-based or processor-based component. For simplicity and ease of illustration, FIG. 2 depicts only one mobile device 206. In practice, however, the system 200 is suitably configured to support a plurality of mobile devices 206, where the patient or user owns or operates at least one of the supported mobile devices 206. An exemplary embodiment of a device suitable for implementation as the mobile device 206 is described below with reference to FIG. 3.

The remainder of this description assumes that the mobile device 206 is a smartphone used by the particular patient. To this end, the configuration and general functionality of the mobile device 206 can be substantially consistent with conventional smartphone design. In this regard, a suitably designed mobile app is installed on the mobile device 206 to allow the patient to receive, view, and interact with messages and notifications provided by the system. The mobile app installed on the mobile device 206 can also be utilized to provide relevant data to other systems (not illustrated) for storage and analysis. For example, the mobile app can manage and upload the following information, without limitation: calendar data (time of day, day of the week, month, season, etc.); user profile data; GPS data that indicates the geographic position of the mobile device 206; map or navigation data associated with operation of the mobile device 206; user-entered meal consumption, food content, and/or food ingredient data; user-entered carbohydrate data; user-entered exercise-related data; user-entered medication-related data; user response data associated with the receipt of glycemic insight messages; user feedback related to glycemic insight messages; accelerometer data; contacts list information; web browser data; consumer purchasing data; and the like.

In certain embodiments, the insulin infusion device 202 is a portable patient-worn or patient-carried component that is operated to deliver insulin into the body of the patient via, for example, an infusion set. In accordance with certain exemplary embodiments, each insulin infusion device 202 supported by the system 200 is implemented as a computer-based or processor-based component. For simplicity and ease of illustration, FIG. 2 depicts only one insulin infusion device 202. In practice, however, the system 200 is suitably configured to support a plurality of insulin infusion device 202, wherein each patient or user owns or operates at least one of the insulin infusion devices 202. An exemplary embodiment of a device suitable for implementation as the insulin infusion device 202 is described below with reference to FIG. 3.

The system 200 obtains input data from one or more sources, which may include various diabetes management devices (an insulin infusion device, a continuous glucose monitoring device, a glucose sensor, a monitor device, or the like). In this regard, the insulin infusion device 202 represents a source of input data for the system 200. In certain embodiments, the insulin infusion device 202 provides data that is associated with its operation, status, insulin delivery events, and the like. As mentioned previously, relevant data generated or collected by the insulin infusion device 202 can be transmitted directly or indirectly to other components of the system including the data uploader component 209, depending on the particular implementation of the system 200. The particular type of data provided by the insulin infusion device 202 is described in more detail below.

The patient or user can own or operate the blood glucose meter 207. The blood glucose meter 207 is configured to measure the blood glucose level of a user by analyzing a blood sample. For example, the blood glucose meter 207 may include a receptacle for receiving a blood sample test strip. In this regard, the user inserts a test strip into the blood glucose meter 207, which analyzes the sample and displays a blood glucose level corresponding to the test strip sample. The blood glucose meter 207 may be configured to communicate the measured blood glucose level to the insulin infusion device 202 (e.g., for storage and processing), to the mobile device 206, or to the data uploader component 209. In some scenarios, the patient is responsible for entering each blood glucose measurement into the insulin infusion device 202. The measured blood glucose data can be provided to any components of the system for analysis.

The glucose sensor 204 can be owned or operated by the patient or user. The glucose sensor 204 is suitably configured to measure a glucose level (e.g., an interstitial glucose level) of the patient in real time. The glucose sensor 204 may include a wireless transmitter that facilitates transmission of the sensor glucose data to other devices, such as the insulin infusion device 202 or the data uploader component 209 or other components of the system, where the sensor glucose data can be received for further processing.

Depending on the particular embodiment and application, the system 200 can include or cooperate with other devices, systems, and sources of input data. Devices within the system 200 may be configured to support the transmission of data to various external devices, such as, without limitation: a stationary monitor device, such as a bedside monitor or a piece of hospital monitoring equipment; a portable computer, such as a laptop PC, a palmtop PC, or a tablet PC; a stationary computer, such as a desktop PC; a personal digital assistant, which may also be a portable email device; one or more additional computing devices or databases; or the like. The above list of possible external devices is not exhaustive, and an implementation of the system 200 can be designed to accommodate communication with other systems, equipment, computing devices, components, and elements that are external to the system 200. For example, in certain embodiments the system 200 includes one or more sources of contextual information or data, which may include, without limitation: activity tracker devices; meal logging devices or apps; mood tracking devices or apps; and the like.

The system 200 includes a local infusion system having one or more local devices configured to wirelessly communicate with each other. This description may refer to the local infusion system as a “personal area network” or a “body area network” of its constituent devices. Local devices may be configured to transmit and receive local communications within the local infusion system, where such local communications are transmitted and received in accordance with one or more specified local data communication protocols. For example, local communications may be exchanged between local devices using one or more wireless data communication protocols (which may leverage RF, infrared, magnetic induction, or other wireless techniques) and/or using one or more wired data communication protocols. Thus, one or more of the local devices can be considered to be a wireless medical device in the context of this description. The local infusion system may be flexibly configured such that any given local device can communicate with any other local device, and a communication link or path between two local devices may be unidirectional or bidirectional. FIG. 2 depicts an exemplary embodiment where each communication link or path is bidirectional (represented by double headed arrows).

Moreover, the local devices in the local infusion system may be capable of communication (unidirectional or bidirectional) with one or more “external” devices that are not considered to be part of the local infusion system. The manner in which a given local device within the local infusion system communicates with a given external device may vary depending upon the particular configuration of the system 200, the characteristics of the local device, and the characteristics of the external device. For example, data may be routed between the local infusion system and an external device using one data communication network, using a plurality of data communication networks, using a direct wireless or wired connection, or the like.

As mentioned above, the system 200 includes or cooperates with computer-based and/or processor-based components having suitably configured hardware and software written to perform the functions and methods needed to support the features described herein. For example, the insulin infusion device 202, the mobile device 206, the blood glucose meter 207 and the data uploader component 209 can each be realized as an electronic processor-based component.

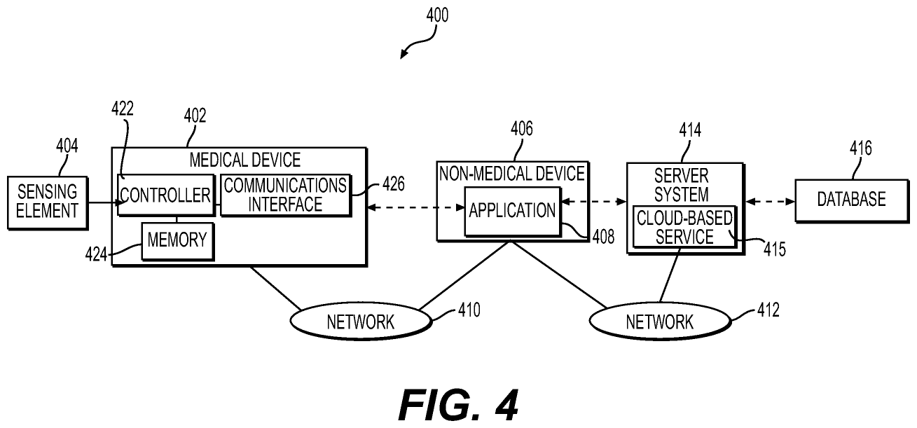

An exemplary embodiment of a device suitable for implementing the various components of the system will described below with reference to FIG. 3. In this regard, FIG. 3 is a simplified block diagram representation of an exemplary embodiment of a computer-based or processor-based device 300 that is suitable for deployment in the system shown in FIGS. 1 and 2. In this regard, each of the devices or components that are described above with reference to FIGS. 1-2 can be realized as a computer-based or processor-based device/component 300. In addition, each of the devices or components that are described below with reference to FIGS. 4-7 can also be realized as a computer-based or processor-based device/component 300.

The illustrated embodiment of the device 300 is intended to be a high-level and generic representation of one suitable platform. In this regard, any of the computer-based or processor-based components of the system 200 can utilize the architecture of the device 300. The illustrated embodiment of the device 300 generally includes, without limitation: at least one processor 302; a suitable amount of memory 304; device-specific hardware, software, firmware, and/or features 306; a user interface 308; a communication module 310; and a display element 311. Of course, an implementation of the device 300 may include additional elements, components, modules, and functionality configured to support various features that are unrelated to the subject matter described here. For example, the device 300 may include certain features and elements to support conventional functions that might be related to the particular implementation and deployment of the device 300. In practice, the elements of the device 300 may be coupled together via a bus or any suitable interconnection architecture 301.

The processor 302 may be implemented or performed with a general-purpose processor, a content addressable memory, a digital signal processor, an application specific integrated circuit, a field programmable gate array, any suitable programmable logic device, discrete gate or transistor logic, discrete hardware components, or any combination designed to perform the functions described here. Moreover, the processor 302 may be implemented as a combination of computing devices, e.g., a combination of a digital signal processor and a microprocessor, a plurality of microprocessors, one or more microprocessors in conjunction with a digital signal processor core, or any other such configuration.

The memory 304 may be realized as RAM memory, flash memory, EPROM memory, EEPROM memory, registers, a hard disk, a removable disk, a CD-ROM, or any other form of storage medium known in the art. In this regard, the memory 304 can be coupled to the processor 302 such that the processor 302 can read information from, and write information to, the memory 304. In the alternative, the memory 304 may be integral to the processor 302. As an example, the processor 302 and the memory 304 may reside in an ASIC. At least a portion of the memory 304 can be realized as a computer storage medium, e.g., a tangible computer-readable medium having computer-executable instructions stored thereon. The computer-executable instructions, when read and executed by the processor 302, cause the device 300 to perform certain tasks, operations, functions, and processes that are specific to the particular embodiment. In this regard, the memory 304 may represent one suitable implementation of such computer-readable media. Alternatively, or additionally, the device 300 could receive and cooperate with computer-readable media (not separately shown) that is realized as a portable or mobile component or platform, e.g., a portable hard drive, a USB flash drive, an optical disc, or the like.

The device-specific hardware, software, firmware, and features 306 may vary from one embodiment of the device 300 to another. For example, the device-specific hardware, software, firmware, and features 306 will support: smartphone functions and features when the device 300 is realized as a mobile telephone; conventional personal computer functions and features if the device 300 is realized as a laptop or tablet computer; insulin pump operations when the device 300 is realized as an insulin infusion device; etc. In practice, certain portions or aspects of the device-specific hardware, software, firmware, and features 306 may be implemented in one or more of the other blocks depicted in FIGS. 3 and 4.

The user interface 308 may include or cooperate with various features to allow a user to interact with the device 300. Accordingly, the user interface 308 may include various human-to-machine interfaces, e.g., a keypad, keys, a keyboard, buttons, switches, knobs, a touchpad, a joystick, a pointing device, a virtual writing tablet, a touch screen, a microphone, or any device, component, or function that enables the user to select options, input information, or otherwise control the operation of the device 300. The user interface 308 may include one or more graphical user interface (GUI) control elements that enable a user to manipulate or otherwise interact with an application via the display element 311.

The communication module 310 facilitates data communication between the device 300 and other components as needed during the operation of the device 300. In the context of this description, the communication module 310 can be employed to transmit or stream device-related control data, patient-related data, device-related status or operational data, glycemic insight messages and notifications, and the like. It should be appreciated that the particular configuration and functionality of the communication module 310 can vary depending on the hardware platform and specific implementation of the device 300. Accordingly, the communication module 310 is utilized to obtain input data from various sources, and to send output data to other components or devices that are described above with reference to FIGS. 1 and 2. In practice, an embodiment of the device 300 may support wireless data communication and/or wired data communication, using various data communication protocols. For example, the communication module 310 could support one or more wireless data communication protocols, techniques, or methodologies, including, without limitation: RF; IrDA (infrared); Bluetooth®; ZigBee® (and other variants of the IEEE 802.15 protocol); IEEE 802.11 (any variation); IEEE 802.16 (WiMAX or any other variation); Direct Sequence Spread Spectrum; Frequency Hopping Spread Spectrum; cellular/wireless/cordless telecommunication protocols; wireless home network communication protocols; paging network protocols; magnetic induction; satellite data communication protocols; wireless hospital or health care facility network protocols such as those operating in the WMTS bands; GPRS; and proprietary wireless data communication protocols such as variants of Wireless USB. Moreover, the communication module 310 could support one or more wired/cabled data communication protocols, including, without limitation: Ethernet; powerline; home network communication protocols; USB; IEEE 2394 (Firewire); hospital network communication protocols; and proprietary data communication protocols. In one particular implementation, the communication module 310 includes a far-field communication module and a body area network communication module, and a controller (not illustrated).

The far-field communication module includes various far-field communication interfaces that can be used to communicate electromagnetic signals to other devices that are part of a body area network. In this non-limiting example, various far-field communication interfaces can include, but are not limited to, a Bluetooth low energy (BLE®) communication interface, a classical Bluetooth® (BT) communication interface, a wireless local area network (WLAN) communication interface (e.g., a Wi-Fi interface), and a cellular communication interface. The above-mentioned communication interfaces can comply with any known standards. For example, the BLE® communication interface can communicate in compliance with any Bluetooth® release (e.g., versions 1.0 through 5.1), and any physical (PHY) layer specifications defined therein. For instance, Bluetooth® 5.0 includes three PHY layer variations called LE 1M, LE 2M and LE Coded. Each PHY variant has its own particular characteristics and was designed with specific aims in mind. As another non-limiting example, the BLE® communication interface can communicate in compliance with a Bluetooth® mesh networking protocol (defined in Mesh Profile Specification and Mesh Model Specification which was adopted on Jul. 13, 2017). The Bluetooth® mesh networking protocol is a protocol based upon Bluetooth Low Energy® that allows for many-to-many communication over Bluetooth® radio.

When a signal from a far-field communication interface is transmitted by an antenna, it is attenuated over distance to the point that the signal cannot be effectively detected. This is called far-field transmission, and works well if the signal needs to be transmitted over a long distance. However, far-field communication interfaces can have problems if the wireless communication needs to be very low power and confines to a fairly short distance near body areas. Improper placement of devices close to a human body may result in a detuned antenna input impedance, reduced antenna efficiency, and distorted antenna radiation pattern. Penetration of electromagnetic signals generated by far-field communication interfaces into the human body is another problem because electromagnetic signals can be quickly absorbed and greatly attenuated due to the very conductive body tissues. In addition, interference can be very high due to coexistence of multiple far-field communication interfaces (e.g., BT, BLE®, Wi-Fi, and ZigBee®) that operate in the same frequency band. Power consumption can also limit continuous operation. Lastly, far-field communication interface can present potential security problems because electromagnetic signals can be intercepted and decrypted after propagating into free space.

On the other hand, the body area network communication module includes various near-field communication interfaces that can be used to communicate magnetic signals to other devices that are part of a body area network. In this non-limiting example, various near-field communication interfaces can include, but are not limited to, a near field magnetic inductive (NFMI) radio communication interface, a near-field electromagnetic induction (NFeMI) radio communication interface (not illustrated), a near field communication (NFC) interface, an RFID high-frequency (HF) communication interface, and one or more low power wide area network (LPWAN) communication interfaces. A near-field communication interface can provide a more reliable, a more secure, and a much lower power radio link within, on, and in the immediate proximity of a human body.

For example, NFMI is a short-range wireless technology that communicates using a tightly coupled magnetic field among devices. NFMI enables human body friendly, reliable, secure, and power efficient wireless communication. As used herein, the term “Near-Field Magnetic Induction (NFMI) radio communication system” can refer to a short range wireless physical layer that communicates by coupling a tight, low-power, non-propagating magnetic field between devices. A transmitter coil in one device can modulate a magnetic field which is measured by a receiver coil in another device. To explain further, in NFMI-based communication systems, a modulated signal that is sent out from a transmitter coil is in the form of a magnetic field. This magnetic field induces voltage on the receiving coil, which in turn will be measured by an NFMI receiver. NFMI radio communication systems differ from other wireless communications in that most conventional wireless RF systems use an antenna to generate, transmit, and propagate an electromagnetic wave, where all of the transmission energy is designed to radiate into free space. This type of transmission is referred to as “far-field.” NFMI systems are designed to contain transmission energy within the localized magnetic field. This magnetic field energy resonates around the communication system, but does not radiate into free space. To explain further, the power density of NFMI signals attenuate at a rate inversely proportional to the distance to the sixth power compared to the second power for Bluetooth® signals. This means for the same distance, the power density of NFMI signals is 10000 times weaker than Bluetooth® signals provided that both transmitting power are equal. This type of wireless transmission is referred to as “near-field.” Various modulation schemes used in typical RF communications (e.g., amplitude modulation, phase modulation, and frequency modulation) can also be used in near-field magnetic induction communication system.

As used herein, the term “near-field electromagnetic induction (NFeMI) radio communication interface” can refer to a communication interface that can operate near a human body by means of a combination of a magnetic field and electric field without the use of transversal radiating waves. Such NFEMI systems improve a wearable device's signal link budget and extend their range to a complete human body. Whereas RF wireless communication may be accomplished by propagating an RF plane wave through free space, NFEMI communication utilizes non-propagating quasi-static fields.

As used herein, the term “Near-field communication (NFC)” can refer to a set of communication protocols and data exchange formats that enable two or more electronic devices (e.g., a medical device such as an insulin pump and a portable device such as a smartphone) to establish communication with each other by bringing them within a short separation range of each other (e.g., 2 meters or less). NFC allows one- and two-way communication between endpoints, suitable for many applications. NFC uses electromagnetic induction between two loop antennas (located within each other's near-field) to effectively form an air-core transformer that allows them to exchange information. The NFC interface operates based on similar principles as the NFMI interface 322 and uses the same high-frequency (HF) band. However, NFMI extends the range of NFC (e.g., from a distance of 1-4 inches for NFC, to up to 9 feet for NFMI). At around 13 MHz, NFMI provides a data rate of over 400 Kbps per frequency channel, up to 10 separate frequency channels and 10 sub-channels per frequency channel using time division (e.g., a hundred separate wireless links inside a single WBAN). In one non-limiting implementation, NFC-enabled devices that are described herein can exchange information in accordance with any NFC standards that cover communications protocols and data exchange formats. NFC standards cover communications protocols and data exchange formats and are based on existing radio-frequency identification (RFID) standards including ISO/IEC 14443. The standards include ISO/IEC 18092 and those defined by the NFC Forum. In addition to the NFC Forum, the GSM Association (GSMA) group defined a platform for the deployment of GSMA NFC Standards within mobile handsets.

RFID systems can operate in low frequency (LF), high frequency (HF), and ultra-high frequency (UHF) bands, and thus can be categorized by the frequency band within which they operate: low frequency, high frequency, and ultra-high frequency. In addition, there are also two broad categories of systems—passive and active RFID. The LF band covers frequencies from 30 KHz to 300 KHz (e.g., some LF RFID systems operate at 125 KHz, while others operate at 134 KHz). The RFID HF communication interface 326 can operate in a HF band that ranges from 3 to 30 MHz, with communications ranges between 10 cm and 1 m. There are several HF RFID standards, such as the ISO 15693 standard for tracking items, and the ECMA-340 and ISO/IEC 18092 standards for Near Field Communication (NFC), the ISO/IEC 14443 A and ISO/IEC 14443 standards. The UHF frequency band covers the range from 300 MHz to 3 GHz, and the range of some UHF systems can be as long as 12 m with faster data transfer rates than LF or HF. The UHF frequency band is regulated by a single global standard called the ECPglobal Gen2 (ISO 18000-63) UHF standard.

The low power wide area network (LPWAN) communication interfaces can include interfaces such as a Long Term Evolution for Machines (LTE-M) communication interface (LTE-Cat M1) and/or a narrowband-IoT (NB-IoT) communication interface (not illustrated). NB-IOT and LTE-M are two newer low power wide area (LPWA) technologies that were developed for IOT applications. Both are protocols for low bandwidth cellular communications that connect to the internet devices that need to transmit small amounts of data, with the lower costs (both hardware and subscription) and the higher battery life.

The various communication interfaces mentioned above are non-limiting, and can be implemented in accordance with any known standards including those mentioned above. However, it should be appreciated that the number of communication interfaces that are included as part of the communication module 310 can vary depending on the implementation.

A controller (not illustrated) can be configured to control which ones of the communication interfaces are selected and used by a device or component of the wireless body area network to communicate data with other devices or components that are part of the wireless body area network. For example, the controller is configured to select which one of the communication interfaces is to be used at any particular time and switch between which one of the communication interfaces is enabled and used by a device or component of the wireless body area network to communicate data with other devices or components that are part of the wireless body area network.

Referring again to FIG. 3, the display element 311 is suitably configured to enable the device 300 to render and display various screens, insight messages, notifications, GUIs, GUI control elements, drop down menus, auto-fill fields, text entry fields, message fields, or the like. Of course, the display element 311 may also be utilized for the display of other information during the operation of the device 300, as is well understood. Notably, the specific configuration, operating characteristics, size, resolution, and functionality of the display element 310 can vary depending upon the practical implementation of the device 300. For example, if the device 300 is a laptop computer, then the display element 311 may be a relatively large monitor. Alternatively, if the device 300 is a cellular telephone device (e.g., smartphone), then the display element 311 may be a relatively small integrated display screen, such as a touch-sensitive screen.

FIG. 4 depicts an exemplary embodiment of a patient monitoring system 400. The patient monitoring system 400 includes a medical device 402 that is communicatively coupled to a sensing element 404 that is inserted into the body of a patient or otherwise worn by the patient to obtain measurement data indicative of a physiological condition in the body of the patient, such as a sensed glucose level. The medical device 402 is communicatively coupled to a non-medical device 406 via a communications network 410, with the non-medical device 406 being communicatively coupled to a server system 414 via another communications network 412. In this regard, the non-medical device 406 may function as an intermediary for uploading or otherwise providing measurement data from the medical device 402 to the server system 414. It should be appreciated that FIG. 4 depicts a simplified representation of a patient monitoring system 400 for purposes of explanation and is not intended to limit the subject matter described herein in any way. In particular, although the term “client” is used to describe the roles of the device 406 relative to the medical device 402 and the server system 414, it should be appreciated that in other embodiments, the device 406 may not exhibit the same relationships. For example, in some embodiments, the device 406 can be a client device with respect to the medical device 402 and a server device with respect to the server system 414.

In exemplary embodiments, the non-medical device 406 is realized as a mobile phone, a smartphone, a tablet computer, or other similar mobile electronic device; however, the non-medical device 406 may be realized as any sort of electronic device capable of communicating with the medical device 402 via network 410, such as a laptop or notebook computer, a desktop computer, or the like. In exemplary embodiments, the network 410 is realized as a BLUETOOTH network, a ZIGBEE network, or another suitable personal area network. That said, in some embodiments, the network 410 could be realized as a wireless ad hoc network, a wireless local area network (WLAN), or local area network (LAN). The non-medical device 406 includes or is coupled to a display device, such as a monitor, screen, or another conventional electronic display, capable of graphically presenting data and/or information pertaining to the physiological condition of the patient. The non-medical device 406 also includes or is otherwise associated with a user input device, such as a keyboard, a mouse, a touchscreen, or the like, capable of receiving input data and/or other information from the user of the non-medical device 406.

In exemplary embodiments, a user, such as the patient, the patient's doctor or another healthcare provider, or the like, manipulates the non-medical device 406 to execute a client application 408 that supports communicating with the medical device 402 via the network 410. In this regard, the client application 408 supports establishing a communications session with the medical device 402 on the network 410 and receiving data and/or information from the medical device 402 via the communications session. The medical device 402 may similarly execute a corresponding application or process that supports establishing the communications session with the client application 408. The client application 408 generally represents a set of instructions that is executed by the non-medical device 406 to perform or facilitate the performance of at least some of the processes described herein. Accordingly, the non-medical device 406 generally includes a processing system and a data storage element (or memory) capable of storing programming instructions for execution by the processing system, that, when read and executed, cause the processing system to perform or facilitate performance of the processes, tasks, operations, and/or functions described herein. Depending on the embodiment, the processing system may be implemented using any suitable processing system and/or device, such as, for example, one or more processor devices, central processing units (CPUs), controllers, microprocessors, microcontrollers, processing cores and/or other hardware computing resources configured to support the operation of the processing system described herein. Similarly, the data storage element or memory may be realized as a random-access memory (RAM), read only memory (ROM), flash memory, magnetic or optical mass storage, or any other suitable non-transitory short or long-term data storage or other computer-readable media, and/or any suitable combination thereof

In one or more embodiments, the non-medical device 406 and the medical device 402 establish an association (or pairing) with one another over the network 410 to support subsequently establishing a point-to-point or peer-to-peer communications session between the medical device 402 and the non-medical device 406 via the network 410. For example, in accordance with one embodiment, the network 410 is realized as a BLUETOOTH network, wherein the medical device 402 and the non-medical device 406 are paired with one another (e.g., by obtaining and storing network identification information for one another) based on performing a discovery procedure or another suitable procedure. The pairing information obtained during the discovery procedure may allow either of the medical device 402 or the non-medical device 406 to initiate the establishment of a secure communications session via the network 410.

In one or more exemplary embodiments, the client application 408 is also configured to store or maintain an address and/or other identification information for the server system 414 on the second network 412. In this regard, the second network 412 may be physically and/or logically distinct from the network 410, such as, for example, the Internet, a cellular network, a wide area network (WAN), or the like. In some embodiments, the server system 414 represents a server or other computing device that hosts a cloud-based service 415 and that is configured to receive and analyze or otherwise monitor measurement data, event log data, and potentially other information obtained for the patient associated with the medical device 402. In exemplary embodiments, the server system 414 is coupled to a database 416 configured to store or maintain data associated with individual patients. In practice, the server system 414 may reside at a location that is physically distinct and/or separate from the medical device 402 and the non-medical device 406, such as, for example, at a facility that is owned and/or operated by or otherwise affiliated with a manufacturer of the medical device 402. For purposes of explanation, but without limitation, the server system 414 may alternatively be referred to herein as a server. It should be appreciated that in some embodiments, the server system 414 may be a client device, both a server device and a client device, or neither a server device nor a client device.

Still referring to FIG. 4, the sensing element 404 generally represents the component of the patient monitoring system 400 that is configured to generate, produce, and/or output one or more electrical signals indicative of a physiological condition that is sensed, measured, and/or quantified by the sensing element 404. In this regard, the physiological condition of a patient influences a characteristic of the electrical signal output by the sensing element 404, such that the characteristic of the output signal corresponds to or is otherwise correlative to the physiological condition that the sensing element 404 is sensitive to. In exemplary embodiments, the sensing element 404 is realized as an interstitial glucose sensing element inserted at a location on the body of the patient that generates an output electrical signal having a current (or voltage) associated therewith that is correlative to the interstitial fluid glucose level that is sensed or otherwise measured in the body of the patient by the sensing element 404.

The medical device 402 generally represents the component of the patient monitoring system 400 that is communicatively coupled to the output of the sensing element 404 to receive or otherwise obtain the measurement data samples from the sensing element 404 (e.g., the measured glucose and characteristic impedance values), store or maintain the measurement data samples, and upload or otherwise transmit the measurement data to the server system 414 or server via the non-medical device 406. In one or more embodiments, the medical device 402 is realized as an infusion device 102, 200 configured to deliver a fluid, such as insulin, to the body of the patient. That said, in some other embodiments, the medical device 402 could be a standalone sensing or monitoring device separate and independent from an infusion device (e.g., sensing arrangement 104, 404). It should be noted that although FIG. 4 depicts the medical device 402 and the sensing element 404 as separate components, in practice, the medical device 402 and the sensing element 404 may be integrated or otherwise combined to provide a unitary device that can be worn by the patient.

In exemplary embodiments, the medical device 402 includes a controller 422, a data storage element 424 (or memory), and a communications interface 426. The controller 422 generally represents the hardware, circuitry, logic, firmware and/or other component(s) of the medical device 402 that is coupled to the sensing element 404 to receive the electrical signals output by the sensing element 404 and perform or facilitate performance of various additional tasks, operations, functions and/or processes described herein. Depending on the embodiment, the controller 422 may be implemented or realized with a general purpose processor device, a microprocessor device, a controller, a microcontroller, a state machine, a content addressable memory, an application specific integrated circuit, a field programmable gate array, any suitable programmable logic device, discrete gate or transistor logic, discrete hardware components, or any combination thereof, designed to perform the functions described herein. In some embodiments, the controller 422 includes an analog-to-digital converter (ADC) or another similar sampling arrangement that samples or otherwise converts an output electrical signal received from the sensing element 404 into corresponding digital measurement data value. In other embodiments, the sensing element 404 may incorporate an ADC and output a digital measurement value.

The communications interface 426 generally represents the hardware, circuitry, logic, firmware and/or other components of the medical device 402 that are coupled to the controller 422 for outputting data and/or information from/to the medical device 402 to/from the non-medical device 406. For example, the communications interface 426 may include or otherwise be coupled to one or more transceiver modules capable of supporting wireless communications between the medical device 402 and the non-medical device 406. In exemplary embodiments, the communications interface 426 is realized as a Bluetooth transceiver or adapter configured to support Bluetooth Low Energy (BLE) communications.

In exemplary embodiments, the server system 414 receives, from the non-medical device 406, measurement data values associated with a particular patient (e.g., sensor glucose measurements, acceleration measurements, and the like) that were obtained using the sensing element 404, and the server system 414 stores or otherwise maintains the historical measurement data in the database 416 in association with the patient (e.g., using one or more unique patient identifiers). Additionally, the server system 414 may also receive, from or via the non-medical device 406, meal data or other event log data that may be input or otherwise provided by the patient (e.g., via client application 408) and store or otherwise maintain historical meal data and other historical event or activity data associated with the patient in the database 416. In this regard, the meal data include, for example, a time or timestamp associated with a particular meal event, a meal type or other information indicative of the content or nutritional characteristics of the meal, and an indication of the size associated with the meal. In exemplary embodiments, the server system 414 also receives historical fluid delivery data corresponding to basal or bolus dosages of fluid delivered to the patient by an infusion device 102, 200. For example, the client application 408 may communicate with an infusion device 102, 200 to obtain insulin delivery dosage amounts and corresponding timestamps from the infusion device 102, 200, and then upload the insulin delivery data to the server system 414 for storage in association with the particular patient. The server system 414 may also receive geolocation data and potentially other contextual data associated with a device 402, 406 from the non-medical device 406 and/or client application 408, and store or otherwise maintain the historical operational context data in association with the particular patient. In this regard, one or more of the devices 402, 406 may include a global positioning system (GPS) receiver or similar modules, components or circuitry capable of outputting or otherwise providing data characterizing the geographic location of the respective device 402, 406 in real-time.

The historical patient data may be analyzed by one or more of the server system 414, the non-medical device 406, and/or the medical device 402 to alter or adjust operation of an infusion device 102, 200 to influence fluid delivery in a personalized manner. For example, the patient's historical meal data and corresponding measurement data or other contextual data may be analyzed to predict a future time when the next meal is likely to be consumed by the patient, the likelihood of a future meal event within a specific time period, the likely size or amount of carbohydrates associated with a future meal, the likely type or nutritional content of the future meal, and/or the like. Moreover, the patient's historical measurement data for postprandial periods following historical meal events may be analyzed to model or otherwise characterize the patient's glycemic response to the predicted size and type of meal for the current context (e.g., time of day, day of week, geolocation, etc.). One or more aspects of the infusion device 102, 200 that control or regulate insulin delivery may then be modified or adjusted to proactively account for the patient's likely meal activity and glycemic response.

In one or more exemplary embodiments, the server system 414 utilizes machine learning to determine which combination of historical sensor glucose measurement data, historical delivery data, historical auxiliary measurement data (e.g., historical acceleration measurement data, historical heart rate measurement data, and/or the like), historical event log data, historical geolocation data, and other historical or contextual data are correlated to or predictive of the occurrence of a particular event, activity, or metric for a particular patient, and then determines a corresponding equation, function, or model for calculating the value of the parameter of interest based on that set of input variables. Thus, the model is capable of characterizing or mapping a particular combination of one or more of the current (or recent) sensor glucose measurement data, auxiliary measurement data, delivery data, geographic location, patient behavior or activities, and the like to a value representative of the current probability or likelihood of a particular event or activity or a current value for a parameter of interest. It should be noted that since each patient's physiological response may vary from the rest of the population, the subset of input variables that are predictive of or correlative for a particular patient may vary from other patients. Additionally, the relative weightings applied to the respective variables of that predictive subset may also vary from other patients who may have common predictive subsets, based on differing correlations between a particular input variable and the historical data for that particular patient. It should be noted that any number of different machine learning techniques may be utilized by the server system 414 to determine what input variables are predictive for a current patient of interest, such as, for example, artificial neural networks, genetic programming, support vector machines, Bayesian networks, probabilistic machine learning models, or other Bayesian techniques, fuzzy logic, heuristically derived combinations, or the like.

The insulin infusion device may incorporate or leverage the control algorithms, processing schemes, and operating methodologies (or suitably modified, updated, or customized versions thereof) of the type described in U.S. Pat. No. 9,526,834 and International (PCT) patent publication number WO 2014/035570; the content of these published documents is incorporated herein by reference.

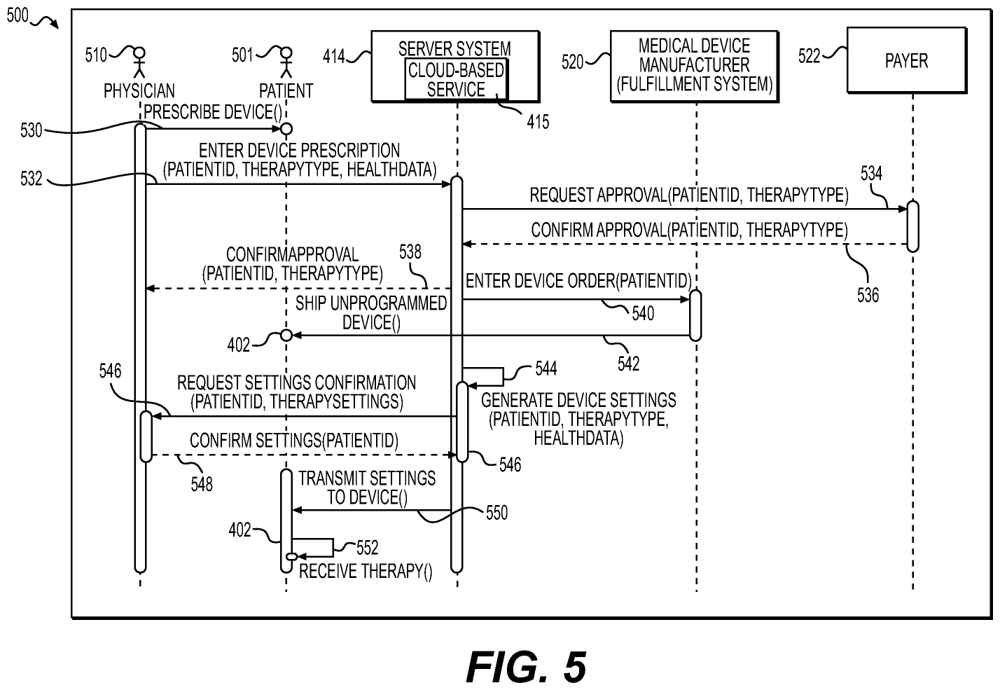

FIG. 5 is a flow diagram that illustrates a medical device configuration method 500 for configuring one or more medical devices 402 that will be provided to a patient 501 in accordance with the disclosed embodiments. For illustrative purposes, the following description of the method 500 may refer to elements mentioned above in connection with FIGS. 1-4, and the method 500 will be described with reference to certain elements of FIG. 4 to illustrate one possible non-limiting implementation; however, it should be appreciated that the method 500 can be implemented in a variety of different system architectures. FIG. 5 shows the interactions that take place involving a prescriber 510 of medical device(s) 402 (e.g., doctor or other health care provider), a server system 414 of a medical device manufacturer that hosts a cloud-based service 415, a fulfillment system 520 of a medical device manufacturer and a payer 522. The device configuration method 500 can be used to automatically and properly configure any number of unprogrammed/unconfigured medical devices that have been prescribed to a particular patient so that those medical devices are configured specifically for that particular patient. For instance, unprogrammed medical devices can refer to a device lacking firmware, whereas unconfigured medical devices can refer to a device programmed with firmware but lacking user-specific configuration parameters.

At 530, the health care provider 510 prescribes a particular medical device 402 to the patient 501. At 532, the health care provider 510 can enter a device prescription that is sent to a service 415 provided by the cloud-based server system 414. The service 415 can be provided, for example, by the manufacturer of the medical devices. The prescription can include various types of information including but not limited to patient identifying information, therapy type and health data for the patient 501 (e.g., device model, patient diagnosis, etc.).

At 534, the service 415 provided by the cloud-based server system 414 communicates a request for approval/authorization to a payer 522 (e.g., insurance company). The approval request can include things such as the patient identifying information, the therapy type, etc. At 536, the payer 522 can confirm approval for the device prescription to the patient 501 and send an approval confirmation to the service 415 provided by the cloud-based server system 414. The approval confirmation can also include information such as the patient identifying information, the therapy type, etc. At 538, the service 415 can send the approval confirmation to the health care provider 510.

At 540, the service 415 provided by the cloud-based server system 414 can submit a device order that includes the patient identifying information to a fulfillment system 520 of the medical device manufacturer. At 542, the fulfillment system 520 of the medical device manufacturer can ship one or more unprogrammed/unconfigured medical devices 402 to the patient 501. At 544, the service 415 provided by the cloud-based server system 414 can generate a parameter load. The parameter load may be a file or some other collection of data that includes, for example, configuration data for therapy settings of the medical device 402 based on information such as patient-specific data, a device prescription and/or a therapy type prescribed for the particular patient 501. In some implementations, the parameter load can be a custom, patient-specific firmware image (e.g., a snapshot or some other representation of device state, including parameter values and settings, at a particular point in time). In some embodiments, the service 415 can process the patient-specific data, the therapy type prescribed for the particular patient and the device prescription to select the appropriate therapy configuration (e.g., algorithms/software/operating modes) for the medical device for the particular patient 501, and can process the patient-specific data (and possibly other inputs like the therapy type prescribed for the particular patient and the device prescription) to calculate, compute, select or otherwise determine detailed therapy parameters and settings, such as, the appropriate device configuration parameters or settings for configuring the medical device of the particular patient 501, and therapy configuration parameters for configuring the medical device of the particular patient 501. The service 415 can then combine all of this information into a parameter load. As such, this parameter load can include, but is not limited to, configuration data for therapy settings including: the appropriate therapy configuration (e.g., algorithms/software/operating modes) for the medical device for the particular patient 501, and therapy parameters and settings for the medical device (e.g., the appropriate device configuration parameters or settings for configuring the medical device of the particular patient 501, and therapy configuration parameters for configuring the medical device of the particular patient 501).

At 546, once the configuration data for therapy settings are generated, the service 415 provided by the cloud-based server system 414 can optionally send a confirmation request to the health care provider 510. Thus, in some embodiments, the parameter load could be generated without review by the health care provider 510. In the example of FIG. 5, the confirmation request can include, for example, information such as the patient identifying information, and the parameter load. At 548, the health care provider 510 can review the confirmation request to ensure that the configuration data for the proposed therapy settings are correct for the particular patient 501. Once the health care provider 510 confirms that the configuration data for the proposed therapy settings are correct for the particular patient 501, the health care provider 510 can send a confirmation message.

At 550, the in response the confirmation message, the service 415 provided by the cloud-based server system 414 can provide the configuration data for therapy settings (e.g., including the parameter load) to the medical device 402 of the patient 501. In some implementations, the cloud-based server system can push the configuration data for therapy settings to the medical device. In some other implementations, the configuration data for therapy settings can be pulled from the cloud-based server system 414 by the medical device. In addition, it should be noted that the cloud-based server system 414 can provide the configuration data for therapy settings to the medical device over either a direct connection or an indirect connection (e.g., via a WLAN connection to the device itself, for example, or via a smartphone application over the internet). Once the device is configured in accordance with the configuration data for therapy settings, the medical device 402 can be activated at 552 and the patient 501 can begin receiving therapy. Depending on the embodiment, the medical device 402 can be activated in various different ways. In some embodiments, the medical device 402 can be activated when the medical device 402 is powered on. In some embodiments, the medical device 402 can be activated when the medical device 402 receives an instruction, command or other user input to start providing therapy to the user. The instruction, command or other user input may be initiated by interaction with a user interface element, such as, a physical button or equivalent on a medical/non-medical device, a graphical user interface element that is displayed on a display device/component included in or coupled to a medical/non-medical device, or any other means that is selectable by the user to activate the medical device 402. For instance, the patient can press one or more buttons according to a prescribed sequence of one or more presses to activate the medical device 402. Alternatively, the patient can interact with one or more user interface elements of the medical device 402 or another non-medical device to activate the medical device 402. In some embodiments, the medical device 402 can be activated when the medical device 402 is placed on or in the patient's body (e.g., installed on or in the patient's body). In some embodiments, the medical device 402 can be activated when the medical device 402 detects deployment, for example, detects that it has been placed on the patient's body. This could be accomplished through various means, including, but not limited to: activation of a cannula insertion mechanism, a signal from a skin contact sensor (e.g., indicating sweat detection, body impedance detection, optical recognition/detection, capacitance detection, or temperature detection), a signal from a mechanical switch between device and patient, a signal from an integrated glucose sensor detecting contact with interstitial fluid, etc. In some embodiments, the medical device 402 can be activated when the medical device 402 when any combination of the above is performed and/or detected.

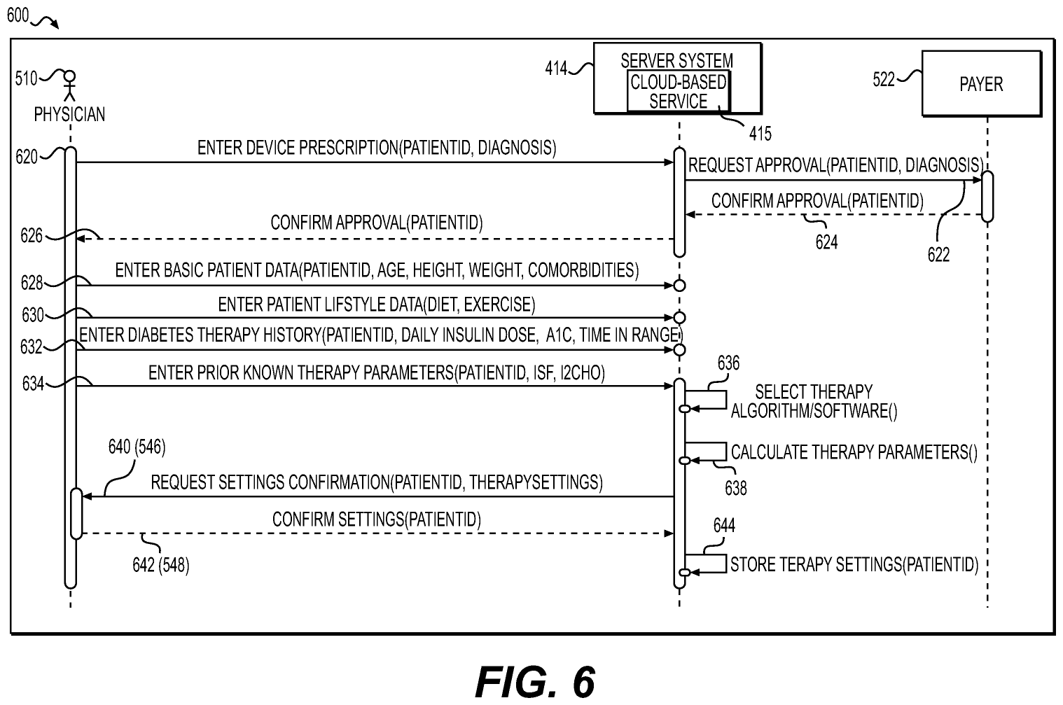

FIG. 6 is a flow diagram that illustrates a settings generation method 600 for generating settings with which to configure one or more medical devices 402 that will be provided to a patient 501 in accordance with the disclosed embodiments. For illustrative purposes, the following description of the method 600 may refer to elements mentioned above in connection with FIGS. 1-5, and the method 600 will be described with reference to certain elements of FIG. 4 to illustrate one possible non-limiting implementation; however, it should be appreciated that the method 600 can be implemented in a variety of different system architectures. FIG. 6 shows the interactions that take place involving a prescriber 510 of medical device(s) 402 (e.g., doctor or other health care provider), a server system 414 of a medical device manufacturer that hosts a cloud-based service 415, and a payer 522. The method 600 can be used in conjunction with the method 500 of FIG. 5. The method 600 can be used to generate one or more settings for properly configuring any number of unprogrammed/unconfigured medical devices for a particular patient 501 so that those medical devices can be configured specifically for that particular patient 501.

At 620, the health care provider 510 can prescribe a particular medical device to the patient 501 by entering, for example, a medical device prescription and therapy type for the patient 501 that is sent to a service 415 provided by the cloud-based server system 414. The medical device prescription for the patient 501 can include, for example, patient identifying information for the patient 501 and other information such as a diagnosis for that particular patient 501. While this is one possible implementation, it should also be noted that this information can also be provided to the service 415 in other ways in other implementations. In some implementations, a therapy type prescribed for the particular patient can be prescribed, for example, when the system generates settings, then the HCP approves them before deploying them to the medical device. This can apply when a patient is prescribed a device or later when a therapy update may be needed due to changes to patient's condition or availability of a new therapy which may not require a new device. In some implementations, a therapy type for the particular patient can be prescribed, for example, when the HCP initiates an update and the system runs an analysis to recommend updated parameters. In some implementations, the therapy type prescribed for the particular patient can be prescribed, for example, when the HCP updates settings based on his/her analysis, but uses the system to download updated parameters that are determined based on his or her analysis. In some implementations, the therapy type prescribed for the particular patient can be prescribed, for example, when the system automatically generates update-based analysis on a periodic basis or when triggered by some measured parameter (e.g., BG levels not being in desired range). In some implementations, the therapy type prescribed for the particular patient can be prescribed, for example, when updated parameters that are downloaded may not require approval from the HCP. The system may download updates directly to patient's medical device.

At 622, the service 415 provided by the cloud-based server system 414 communicates a request for approval to a payer 522 (e.g., insurance company). The request for approval can include things such as the patient 501 identifier, the diagnosis, etc.

At 624, the payer 522 can confirm approval for the device prescription to the patient 501 and send an approval confirmation to the service 415 provided by the cloud-based server system 414. The approval confirmation can also include information such as the patient 501 identifier, etc. At 626, the service 415 provided by the cloud-based server system 414 can send the approval confirmation to the health care provider 510.

At 628 through 634, the health care provider 510 can enter various patient-specific data that can be utilized by the service 415 to generate configuration data for therapy settings for the particular patient 501. The particular order of entering patient-specific data may vary from implementation to implementation. The patient-specific data can include various types of information including, but not limited to, basic patient data (entered at step 628), patient lifestyle data (entered at step 630), information about the medical condition being targeted for therapy and information about medical conditions other than the condition being targeted for therapy (entered at step 632 such as diabetes therapy history), prior known therapy parameters (entered at step 634), etc. Examples of basic patient data can include physical, metabolic or anatomical data about the patient including things such as a patient identifying information, the patient's age, height, weight, information about other medical conditions (or comorbidities), etc. The patient lifestyle data can include things like habits of the patient such as data regarding the patient's diet, data regarding the exercise characteristics of the patient, sleep habits, etc. Information about a medical condition being targeted for therapy and information about medical conditions other than the condition being targeted for therapy, which in the case of diabetes, can include information about the patient's diabetes therapy history (e.g., diabetes treatment details/history) including information such as the patient identifying information, clinical diagnosis, the daily insulin dosages for the patient, the A1C for the patient, the time in range for the patient, therapy goals (e.g., preference for aggressive control of diabetes vs. preference for minimized burden), etc. The prior known therapy parameters can include things such as the patient identifying information, therapy algorithms or device operating modes, insulin sensitivity factor of the patient, insulin to carbohydrate ratio of the patient, insulin delivery rate limits of the patient, basal rate profiles for the patient, target glucose level for the patient, etc.

In general terms, the selection of therapy algorithms and software configuration, as well as the computation of therapy parameters, is a function of the inputs provided by the prescribing health care provider 510. However, in some embodiments, other information such as patient history, statistical data, mathematical models, etc. that are provided by the manufacturer of the medical device can also be utilized to select appropriate therapy algorithms and software configuration, and/or to compute of therapy parameters, configuration data, settings, etc. In addition, or alternatively, in some implementations, a computer-based or software-based system can collect information about a patient from different sources and that information can then be used to compute or look up device configuration data, settings, parameters, etc.

At 636, based on the information entered by the health care provider 510 at steps 620 and 628 through 634, the service 415 can select an appropriate therapy configuration (e.g., algorithms/software/operating modes) for the medical device to be used in configuring the medical device for the particular patient 501.

Each different model of medical device is capable of being configured for different therapy configurations. At least some of the patient-specific data can be used to configure or tune therapy parameters of the therapy algorithm/software to configure the medical device for the particular patient 501. At 638, the service 415 can calculate, compute, select or otherwise determine appropriate detailed therapy parameters and settings (e.g., the appropriate device configuration parameters or settings for configuring the medical device of the particular patient 501, and therapy configuration parameters for configuring the medical device of the particular patient 501). The appropriate therapy parameters and settings can be determined or generated based on at least some of the information entered by the health care provider 510 at steps 628 through 634, and at step 620 (e.g., the therapy type prescribed for the particular patient, and the device prescription). As described above, the service 415 can combine all of this information into a parameter load.

At 640, once the service 415 provided by the cloud-based server system 414 selects an appropriate therapy configuration for the medical device (e.g., an appropriate therapy algorithm/software) and generates appropriate therapy parameters and settings to be used in configuring the medical device, the service 415 may communicate a confirmation request to the health care provider 510. The confirmation request can include information such as the patient identifying information, and configuration data for therapy settings including: the appropriate therapy type/algorithm/software for the particular patient 501, the appropriate device configuration parameters or settings for configuring the medical device of the particular patient 501, and therapy configuration parameters for configuring the medical device of the particular patient 501, etc.

At 642, the health care provider 510 can review the confirmation request to ensure that the suggested therapy settings are appropriate and correct for the particular patient 501. Once the health care provider 510 confirms that the proposed therapy settings are correct for the particular patient 501, the health care provider 510 can send a confirmation message to the service 415 provided by the cloud-based server system 414 (that includes the patient identifying information of the particular patient 501) to confirm that the suggested therapy settings are appropriate and correct for that particular patient 501.

At 644, in response the confirmation message, the service 415 can maintain configuration data for the confirmed therapy settings for the particular patient 501 in a database (not shown) of the server system 414. Some information included in the configuration data for therapy settings (e.g., required for proper configuration of medical devices) may be sensitive. Instead of requiring patients and physicians to store human-readable copies of this information, this process allows a physician to enter all therapy data into a secure online system. The settings generated by this process may be stored and transmitted to a patient's device entirely within encrypted, authenticated channels to protect against unauthorized disclosure.

Although not shown in FIG. 6, the service 415 can retrieve, from the database, and send the configuration data for the confirmed therapy settings (i.e., that are appropriate for the particular patient 501) to the medical device of the patient 501 so that the medical device can be configured in accordance with the configuration data for the therapy settings for the particular patient 501. This can be done before or after the device is shipped or delivered to the patient.

For example, in some implementations, the configuration data for the therapy settings may be programmed into devices by the manufacturer at or prior to shipment to patient. In other words, medical device(s) can be programmed “just-in-time” prior to shipment to the patient. In some other implementations, the configuration data for the therapy settings may be programmed into unconfigured devices after the devices are delivered to patient (e.g., via a direct internet connection or by means of a secondary device such as a smartphone or PC). Alternatively, the configuration data for the therapy settings could be electronically transmitted to the patient in human-readable form for manual entry.

In some implementations, this configuration process may take place many times to populate replacement or alternate devices with the same configuration data. For instance, in some cases, multiple devices may be sent to a patient in a single shipment, either to support alternating use (one device operated while another is charged) or as part of a disposable device model. Alternatively, new devices may be sent to a patient on a recurring basis as existing devices reach their useful life.

These variations may extend the system's behavior in a number of ways. Each device may be programmed by the cloud service with the same therapy configuration, either all at once (upon receipt by the patient) or as each device enters service. Each device may be programmed by the cloud service with an updated therapy configuration as it enters service. The therapy configuration may be updated based on data sent back from the prior device, updates made by the prescribing physician, or other information known to the cloud service. Each new device may receive an updated therapy configuration from the preceding device over a local data network, such as Bluetooth or Wi-Fi. This transfer process may not involve the cloud service. Once the medical device is properly configured and activated, the patient 501 can begin receiving therapy.

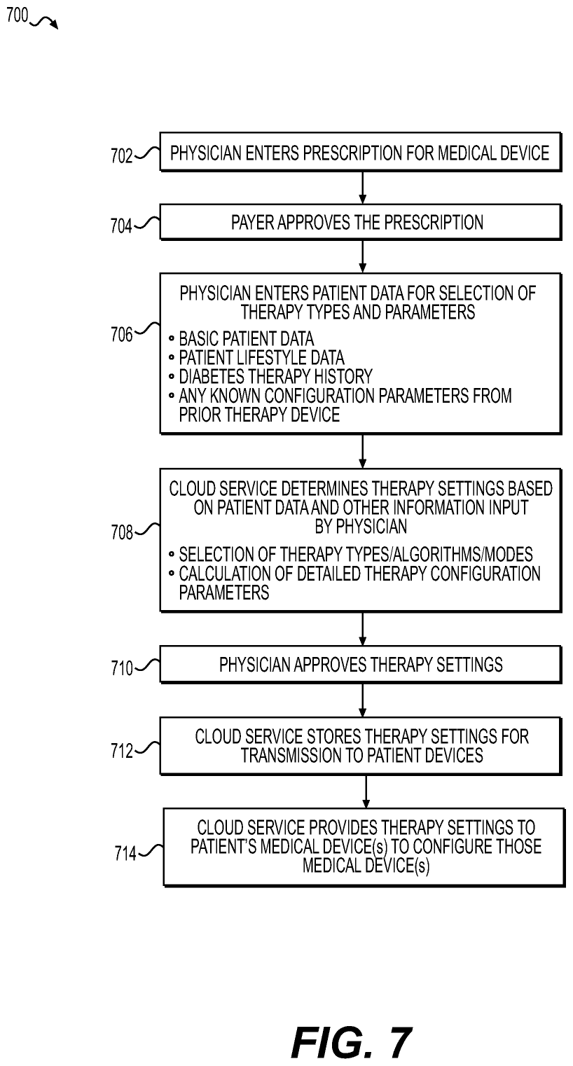

FIG. 7 is a flowchart that illustrates a medical device configuration method 700 for configuring one or more medical devices that will be provided to a patient in accordance with the disclosed embodiments. As a preliminary matter, it should be understood that the method 700 is provided as a non-limiting example. FIG. 7 illustrates one possible embodiment of a prescribing process. In the description of FIG. 7, a particular example is described in which the cloud-based service 415 provided by the cloud-based server system 414 performs certain prescribing actions by interacting with the prescribing physician and the payer described above with reference to FIGS. 1-6.

With reference to method 700, tasks can be added, omitted, and/or performed simultaneously without departing from the scope of the appended claims. It should be appreciated that the method 700 may include any number of additional or alternative tasks, that the tasks shown in FIG. 7 need not be performed in the illustrated order, and that the method 700 may be incorporated into a more comprehensive procedure or process having additional functionality not described in detail herein. Moreover, one or more of the tasks shown in FIG. 7 could potentially be omitted from an embodiment of the method 700 as long as the intended overall functionality remains intact. It should also be understood that the illustrated method 700 can be stopped at any time.

The method 700 is computer-implemented in that various tasks or steps that are performed in connection with the method 700 may be performed by software, hardware, firmware, or any combination thereof. In certain embodiments, some or all tasks of this process, and/or substantially equivalent tasks, are performed by execution of processor-readable instructions stored or included on a processor-readable medium. For illustrative purposes, the following description of the method 700 may refer to elements mentioned above in connection with FIGS. 1-6, and the method 700 will be described with reference to certain elements of FIG. 4 to illustrate one possible non-limiting implementation; however, it should be appreciated that the method 700 can be implemented in a variety of different system architectures. For instance, in the description of FIG. 7 that follows, a cloud-based service 415 provided by a cloud-based server system 414 will be described as performing various acts, tasks or steps, but it should be appreciated that this refers to processing system(s) of these entities executing instructions to perform those various acts, tasks or steps. Depending on the implementation, the method 700 can be performed by an application or service 415 hosted at the cloud-based server system 414.

At 702, the physician enters the initial prescription, and at 704, the payer approves of the prescription. At 706, the physician can enter patient-specific data. The patient-specific data is used for selection of therapy types and parameters. The patient-specific data can include, but is not limited to, basic patient data (e.g., age, height, weight, comorbidities, etc.), patient lifestyle data (e.g., diet and exercise habits, etc.), diabetes therapy history (e.g., total daily dose (TDD) of insulin, A1C, time-in-range, etc.), any known configuration parameters from prior therapy devices (e.g., ISF, I2CHO).

At 708, the cloud-based service 415 provided by the cloud-based server system 414 can determine therapy settings. Step 708 can include selection of the appropriate therapy configuration for the medical device for the particular patient 501, and the calculation of detailed therapy parameters. In some embodiments, the service 415 can process the patient-specific data, the therapy type and the device prescription and select the appropriate therapy configuration (e.g., algorithms/software/operating modes) for the medical device for the particular patient 501, and can process the patient-specific data, the therapy type and the device prescription to calculate or otherwise determine detailed therapy parameters and settings for the medical device, such as, the appropriate device configuration parameters or settings for configuring the medical device of the particular patient 501, and the appropriate therapy configuration parameters for configuring the medical device of the particular patient 501. The service 415 can combine all of this information into a parameter load.

As such, this parameter load can include configuration data for therapy settings including, but is not limited to: the appropriate therapy configuration (e.g., algorithms/software/operating modes) for the medical device for the particular patient 501, the appropriate device configuration parameters or settings for configuring the medical device of the particular patient 501, and the appropriate therapy configuration parameters for configuring the medical device of the particular patient 501.

At 710, the physician can approve the therapy prescription (specified by the parameter load) if it is appropriate/proper, or disapprove the therapy prescription (specified by the parameter load) if it is inappropriate or improper. If the therapy prescription is approved, at 712, the cloud-based service 415 can store the configuration data for therapy settings in storage at the cloud-based server system 414 so that the therapy settings can be transmitted to patient's medical device(s). At 714, the cloud-based service 415 provides configuration data for the approved therapy settings (that are appropriate for the particular patient 501) to the medical device of the patient 501 so that the medical device can be configured in accordance with the configuration data for the therapy settings for the particular patient 501.

In some examples, an insulin infusion device can be configured with patient-specific therapy settings and parameters, for example, before entering an automatic operating mode and starting closed-loop therapy. These therapy settings can include, for example, a basal rate for the specific patient, an insulin sensitivity factor for the specific patient, an insulin to carbohydrate ratio for the specific patient, an active insulin time for the specific patient, a maximum bolus limit for the specific patient, a maximum basal rate for the specific patient, a bolus increment for the specific patient, and a basal increment for the specific patient. An extended warm up period is typically needed to set up these therapy settings and parameters. For instance, one commercially available insulin infusion device requires a minimum of two full days of total daily dose (TDD) values before entering an automatic operating mode and starting closed-loop therapy. This period is sometimes referred to as a long warmup period.

Many patients prefer that their medical devices are easily configurable. In accordance with the disclosed embodiments, systems and methods are provided for configuring therapy settings and parameters of a medical device. These systems and methods can be automated by leveraging patient data including the patient's weight and/or the patient's estimated total daily dose (TDD) of insulin. This patient data may be obtained via a non-medical device (e.g., the patient's smartphone or HCP's computer) or through a cloud-based service during system startup. In accordance with certain embodiments, a patient's estimated TDD of insulin can be determined based on the patient's weight. The TDD can then used to compute various therapy settings for that specific patient. These therapy settings can then be automatically programmed into a medical device during system startup and used, for example, by a closed-loop algorithm in lieu of therapy settings that would otherwise be determined after long warmup period (e.g., two-day TDD requirement).

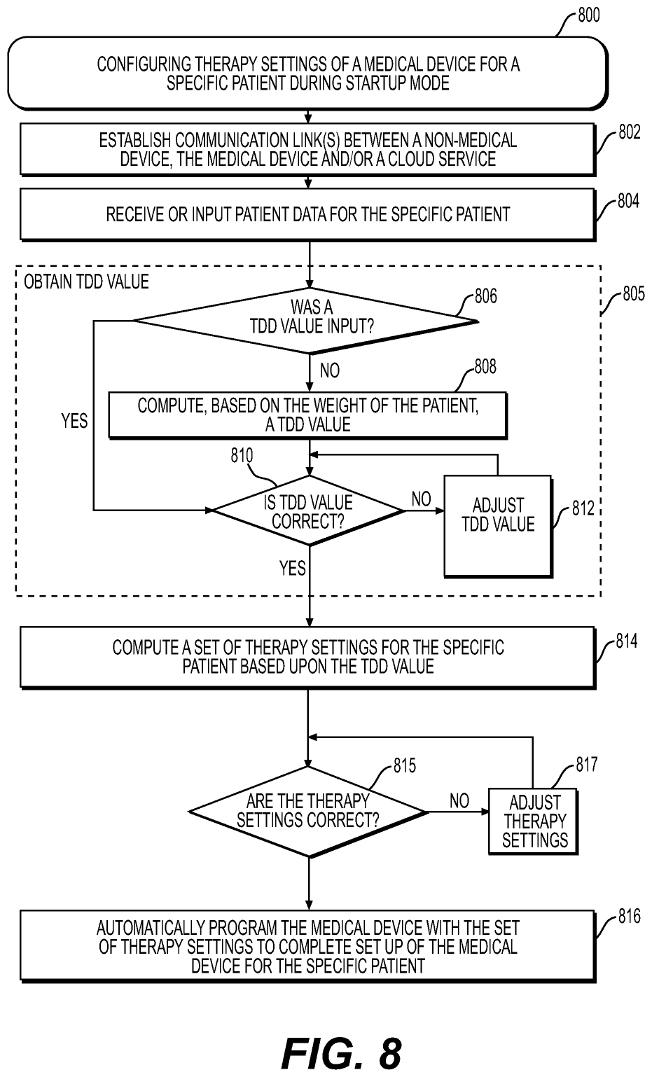

The disclosed embodiments can allow for the medical device to enter an automatic operating mode and begin closed-loop therapy while bypassing the conventional long warmup period. This can allow patients to simplify the system startup process and start closed-loop therapy sooner. To help simplify the process of configuring insulin infusion devices, cloud services and mobile devices, such as smartphones, can be leveraged to help improve the setup process. This can be particularly helpful, for example, with therapy devices that do not include a local graphical user interface (e.g., patch style pumps). Various embodiments will now be described below with reference to FIGS. 8-11.

FIG. 8 is a flowchart that illustrates a method 800 for configuring therapy settings of a medical device 402 for a specific patient during startup mode of the medical device 402 in accordance with the disclosed embodiments. As a preliminary matter, it should be understood that the method 800 is provided as a non-limiting example that illustrates one possible embodiment of a method 800 for configuring therapy settings. With reference to method 800, tasks can be added, omitted, and/or performed simultaneously without departing from the scope of the appended claims. It should be appreciated that the method 800 may include any number of additional or alternative tasks, that the tasks shown in FIG. 8 need not be performed in the illustrated order, and that the method 800 may be incorporated into a more comprehensive procedure or process having additional functionality not described in detail herein. Moreover, one or more of the tasks shown in FIG. 8 could potentially be omitted from an embodiment of the method 800 as long as the intended overall functionality remains intact. It should also be understood that the illustrated method 800 can be stopped at any time.

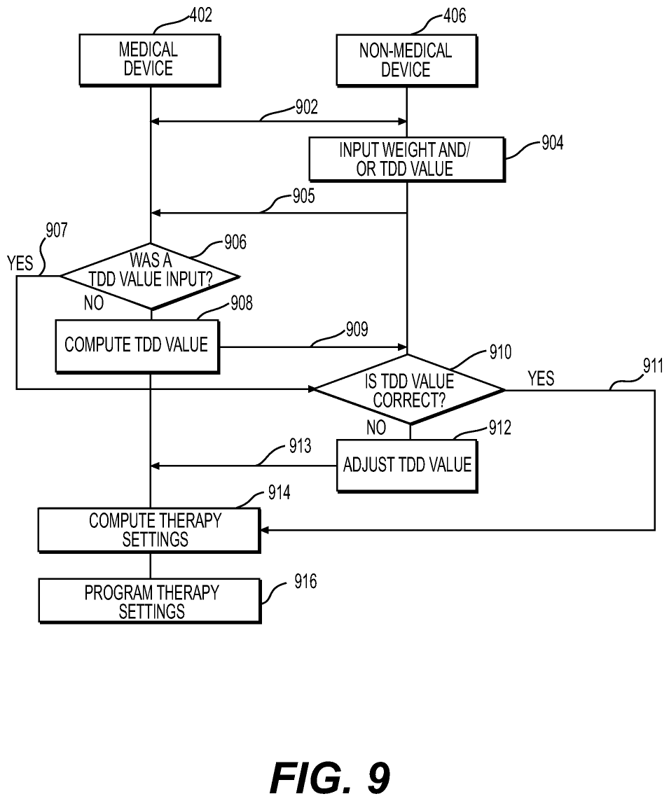

The method 800 is computer-implemented in that various tasks or steps of the method 800 may be performed by software, hardware, firmware, or any combination thereof. In certain embodiments, some or all tasks of this process, and/or substantially equivalent tasks, are performed by execution of processor-readable instructions stored or included on a processor-readable medium. For illustrative purposes, the following description of the method 800 may refer to elements mentioned above in connection with FIGS. 1-6. The method 800 will be described with reference to certain elements of FIG. 4 to illustrate one possible non-limiting implementation; however, it should be appreciated that the method 800 can be implemented in a variety of different system architectures. For instance, in the description of FIG. 8 that follows, a medical device 402, a non-medical device 406, and/or a cloud-based service 415 provided by a cloud-based server system 414 will be described as performing various acts, tasks or steps, but it should be appreciated that this refers to processing system(s) of these entities executing instructions to perform those various acts, tasks or steps. Depending on the implementation, the method 800 can be performed by the medical device 402, the non-medical device 406 and/or the cloud-based service 415 provided by the cloud-based server system 414. Specific implementations will be described below with reference to FIGS. 9-11.

In some embodiments, the method 800 starts at 802, where a non-medical device 406 establishes a communication link with the medical device 402 and/or the server system 414 that hosts the cloud-based service 415. Alternatively, in another embodiment, the medical device 402 can establish a communication link with the non-medical device 406 and/or the server system 414. In another embodiment, the server system 414 can establish a communication link with the medical device 402 and/or the non-medical device 406. In any of these embodiments, the communication link can be established automatically or in response to action by the specific patient that causes these communication links to be established.

At 804, patient data for a specific patient can be input, and optionally sent to and received by one or more of the medical device 802, the non-medical device 406 and/or the cloud-based service 815 at the server system 814. For example, the patient data can be sent from the cloud-based service 815 to the medical device 402 and/or to the non-medical device 406, or from the non-medical device 406 to the cloud-based service 815 and/or to the medical device 802. In some embodiments, the patient data can be input using a graphical user interface of the non-medical device 406 (e.g., a device of the patient). In other embodiments, the patient data can be entered via a health care provider via another non-medical device, stored at the server system 414, and then retrieved from the server system 414 by the non-medical device 406.

The patient data can include one or more of the body weight of the specific patient and/or a value (units) of a TDD of insulin for the specific patient. In some embodiments, the patient data that is input at 804 can also include other product and/or patient health information. Product information can include things such as: firmware version to be installed for the medical device 402; operating parameters for the medical device 402; date and time services for synchronizing devices; a set of features to be activated, for example, CGM integration, closed-loop therapy, remote operation of the infusion device, etc. Patient health information can include things such as one or more of: age of the specific patient, age of the specific patient at the time of diabetes diagnosis, body mass index (BMI) of the specific patient, gender of the specific patient, a prescription from the patient's physician (e.g., physician approved therapy settings for the specific patient), etc.

At 805, a value (units) of a TDD of insulin for the specific patient can be obtained. How the value is obtained can vary depending on the implementation. In some cases, the value may be input at 804, whereas in other cases, the value may be computed based on other patient data and/or adjusted by a user. At 806, it can be determined whether a value of the total daily dose was input as part of the patient data. When a value of the TDD was input as part of the patient data (at 804), the method 800 can proceed to 810.

When a value of the TDD was not input as part of the patient data (at 804), the method 800 can proceed to 808, where the value of the TDD can be computed using the weight of the patient that was input at 804. When the patient's weight is within a lower range (e.g., between 15 kilograms and 38 kilograms), the computation at 808 can be performed by scaling the patient's weight according to a constant scaling factor.

For example, in one embodiment, the weight of the patient can be converted to the value of the TDD using a conversion factor as TDD is a function of the patient's weight. For instance, in one implementation, the weight of the patient can be input in pounds (lb.) and can converted to the value of the TDD by multiplying the weight by a conversion factor or a constant value. In another implementation, the weight of the patient can be input in kilograms (kg) and can converted to the value of the TDD by multiplying the weight by a conversion factor or a constant value.

When the patient's weight is within a higher range (e.g., between 38 kilograms and 200 kilograms), the computation at 808 can be performed by scaling the patient's weight according to a scaling formula that is dependent on the patient. Depending on the implementation, the value of the TDD can be computed (at 808) by the non-medical device 406, the cloud-based service 415 at the server system 414, or the medical device 402. When the value of the TDD is computed (at 808) by the cloud-based service 415 or the medical device 402, it can be sent back to the non-medical device 406 for review or confirmation.

At 810, it can be confirmed whether the value of the TDD, which can be a value input by a user (e.g., patient or HCP) at 804 or a value that was computed as a function of the patient's weight at 808, should be used to compute therapy settings (at 814), or changed/adjusted (at 812).

In some embodiments, at 810, a GUI can be displayed at the non-medical device 406 that includes an interface element that requests confirmation of the value by a user (e.g., the specific patient or their health care provider). This way, the user can confirm whether the value of the TDD (from 804 or 808) is acceptable and should be used to compute therapy settings. A user of the non-medical device 406 can confirm the value of the TDD (at 810) or adjust the value of the TDD (at 812).

When the user inputs information (at 810) to confirm that the value of the TDD is acceptable and should be used to compute therapy settings, the method proceeds to 814. By contrast, when the user inputs information (at 810) to indicate that the computed value of the TDD (from 804 or 808) is not acceptable and should not be used to compute therapy settings, the method proceeds to 812.

At 812, the value of the TDD can be adjusted/changed. Depending on the embodiment, the value of the TDD can be adjusted/changed either based on a user input or algorithmically. For instance, in one embodiment, the user (e.g., patient or HCP) can input an adjusted value of the TDD, and the method 800 loops back to 810 for confirmation that the adjusted value of the TDD should be used to compute therapy settings. In another embodiment, at 812, an algorithm, that can be implemented at the non-medical device 406 (e.g. smart phone or other device), the cloud-based service 415 at the server system 414 (e.g., a server), or the medical device 402, can adjust the value of the TDD, and the method 800 loops back to 810 for confirmation that the adjusted value of the TDD should be used to compute therapy settings. In some embodiments, the algorithm can employ various artificial intelligence and/or machine learning technologies to determine whether the value of the TDD is appropriate for use in computing the therapy settings, and if not, the algorithm can adjust value of the TDD to a value that is acceptable for use in computing the therapy settings.

At 814, the value of the TDD, which can be a value input by a user (e.g., patient or HCP) at 804 or a value that was computed at 808 or an adjusted value that was set or computed at 812, can be used to compute therapy settings. Depending on the implementation, the therapy settings can be computed (at 808) by the non-medical device 406, the cloud-based service 415 at the server system 414, or the medical device 402. For example, in one embodiment, the non-medical device 406 can compute the therapy settings and communicate them to the medical device 402. In another embodiment, the cloud-based service 415 can compute the therapy settings, and communicate the therapy settings to the medical device 402. In another embodiment, the cloud-based service 415 can compute the therapy settings, communicate them to the non-medical device 406, and the non-medical device 406 can communicate the therapy settings to the medical device 402. In another embodiment, the medical device 402 can compute the therapy settings locally based on the value of the TDD.

The set of therapy settings for the specific patient that are computed at 814 can include, but are not limited to, for example: a basal rate for the specific patient, an insulin sensitivity factor for the specific patient, an insulin to carbohydrate ratio for the specific patient, an active insulin time for the specific patient, a maximum bolus limit for the specific patient, a maximum basal rate for the specific patient, a bolus increment for the specific patient, a basal increment for the specific patient, etc. In some implementations, certain parameters (e.g., insulin sensitivity factor and insulin to carbohydrate ratio) can be a set of values that are computed and applied over a specified period of time, such as a twenty-four hour period. In other implementations, certain parameters (e.g., insulin sensitivity factor and insulin to carbohydrate ratio) can be single values that are computed and applied over a specified period of time, such as a twenty-four hour period.

In addition, in some embodiments, product information and/or patient health information can also be communicated to the medical device 402 at 814. Product information can include, for example, a firmware version to be installed for the medical device 402; operating parameters for the medical device 402; date and time services for synchronizing devices, etc. Patient health information can include, for example, age of the specific patient, age of the specific patient at the time of diabetes diagnosis, body mass index (BMI) of the specific patient, gender of the specific patient, the weight of the patient, the value of the TDD, a basal pattern with at least one time segment, active insulin time (hours), BG target range, a prescription from the patient's physician (e.g., physician approved therapy settings for the specific patient, etc.).

In some embodiments, after the therapy settings are computed at 814, the method can proceed to 815, where it can be confirmed whether the computed therapy settings are correct or appropriate. In other words, the therapy settings can be sent, for example, to the non-medical device 406 for confirmation or adjustment by a user (e.g., patient or HCP) before they are used to automatically program the medical device 402 (at 816). Depending on the implementation, the computed therapy settings (from 814) can be adjusted/changed (at 817) algorithmically prior to being used to automatically program the medical device 402, or adjusted/changed (at 817) by a user prior to being used to automatically program the medical device 402. For example, in one embodiment, a GUI can be displayed at the non-medical device 406 that includes an interface element that requests confirmation of the therapy settings by a user (e.g., the specific patient or a health care provider). This way, the user can confirm whether the therapy settings (from 814) are acceptable or whether they need to be changed/adjusted, in which case the user can change/adjust one or more of the therapy settings. When the user inputs information to indicate that the computed therapy settings (from 814) are not acceptable and should not be used to program the medical device 402, the user can input one or more adjusted therapy settings, and the method 800 then proceeds to 816. When the user inputs information to confirm that the therapy settings are acceptable, therapy settings can be used at 816 without being adjusted or changed. In another embodiment, an algorithm can confirm (at 815) whether the computed therapy settings (from 814) are to be used in programming therapy settings (at 816). The algorithm can employ various artificial intelligence and/or machine learning technologies to determine whether the computed therapy settings (from 814) are appropriate for use as the programming therapy settings (at 816), and if not, at 817, can adjust/change one or more value(s) of the computed therapy settings (from 814) to other value(s) that are acceptable for use in programming therapy settings (at 816).

At 816, a processor device or controller of the medical device 402 can be automatically programmed with the set of therapy settings for the specific patient to complete set up of the medical device 402 so that the medical device is properly configured for the specific patient. The set of therapy settings for the specific patient that are used to automatically program the medical device 402 can be either the set of therapy settings that were computed at 814, or a set of adjusted therapy settings that were adjusted/changed after being computed at 814.

FIG. 9 is a flow diagram that illustrates a method 900 for configuring therapy settings of a medical device 402 for a specific patient during startup mode of the medical device 402 in accordance with the disclosed embodiments. The method 900 starts at 902, where a non-medical device 406 establishes a communication link with the medical device 402. Alternatively, in another embodiment, the medical device 402 can establish a communication link with the non-medical device 406. In either of these embodiments, the communication link can be established automatically or in response to action by the specific patient that causes these communication links to be established.

At 904, patient data can be input via the non-medical device 406 (e.g., a device of the patient or a health care provider). In some embodiments, the patient data can be input using a graphical user interface of the non-medical device 406 (e.g., during a pairing process with the medical device 402). In another embodiment, the patient data can be entered via a health care provider via another non-medical device. The patient data can include one or more of the body weight of the specific patient and/or a value (Units) of a TDD of insulin for the specific patient. As described above with reference to FIG. 8, in some embodiments, the patient data that is input at 904 can also include other product and/or patient health information.

At 905, the patient data can be sent from the non-medical device 406 to the medical device 402. At 906, the medical device 402 can determine whether a value of the TDD was input as part of the patient data. When the medical device 402 determines that a value of the TDD was input (as shown by arrow 907) as part of the patient data (at 904), the method 900 can proceed to 910.

When the medical device 402 determines that a value of the TDD was not input as part of the patient data (at 904), the method 900 can proceed to 908, where the medical device 402 can compute a value of the TDD using the weight of the patient that was input at 904. This computation can be performed as described above with reference to FIG. 8. After the value of the TDD is computed (at 908) by the medical device 402, the medical device 402 can send the value back to the non-medical device 406 for review or confirmation by a user (e.g., patient or HCP).

At 910, the non-medical device 406 can confirm whether the value of the TDD, which can be a value input by a user (e.g., patient or HCP) at 904 or a value that was computed as a function of the patient's weight at 908, should be used in computing therapy settings (at 914), or changed/adjusted (at 912) either algorithmically or by a user.

In some embodiments, at 910, a GUI can be displayed at the non-medical device 406 that includes an interface element that requests confirmation of the value by a user (e.g., the specific patient or a health care provider). This way, the user can confirm whether the value of the TDD (from 904 or 908) is acceptable and should be used to compute therapy settings (at 914) or whether the computed value of the TDD should be adjusted/changed at 912. A user of the non-medical device 406 can confirm the value of the TDD (at 910) or adjust the value of the TDD (at 912).

When the user inputs information (at 910) to confirm that the value of the TDD (from 904 or 908) is acceptable and should be used to compute therapy settings, the value of the TDD can be sent (at 911) to the medical device 402. In addition, in some embodiments, product and/or patient health information can also be communicated to the medical device 402 at 911 (or alternatively at 905 or 913).

By contrast, when the user inputs information (at 910) to indicate that the value of the TDD (from 904 or 908) is not acceptable and should not be used to compute therapy settings, the method proceeds to 912, where the value of the TDD can be adjusted/changed. Depending on the embodiment, the value of the TDD can be adjusted/changed either based on a user input or algorithmically. For instance, in one embodiment, at 912, the user (e.g., patient or HCP) can input an adjusted value of the TDD that should be used to compute therapy settings. In another embodiment, at 912, an algorithm at the non-medical device 406 can adjust/change the value of the TDD to an adjusted value of the TDD that should be used to compute therapy settings. In some embodiments, the algorithm can employ various artificial intelligence and/or machine learning technologies to determine whether the value of the TDD is appropriate for use in computing the therapy settings, and if not, the algorithm can adjust value of the TDD to a value that is acceptable for use in computing the therapy settings. At 913, the adjusted value can be sent from the non-medical device 406 to the medical device 402. In addition, as described above with reference to FIG. 8, in some embodiments, the non-medical device 406 can also communicate product and/or patient health information to the medical device 402 at 913 (or alternatively at 905 or 911 as described above).

At 914, the medical device 402 can compute therapy settings based on the value of the TDD, which can be a value input by a user (e.g., patient or HCP) at 904, a value that was computed at 908 or an adjusted value that was adjusted/changed at 912. The set of therapy settings for the specific patient that are computed at 914 can include, but are not limited to, for example: a basal rate, an insulin sensitivity factor for the specific patient, an insulin to carbohydrate ratio for the specific patient, an active insulin time for the specific patient, a maximum bolus limit for the specific patient, a maximum basal rate for the specific patient, a bolus increment for the specific patient, a basal increment for the specific patient, etc. In one non-limiting implementation, the therapy settings can be computed as described above with reference to FIG. 8.

Although not illustrated in FIG. 9, in one embodiment, after the therapy settings are computed at 914, the therapy settings can be sent to the non-medical device 406 for confirmation or adjustment by the user (e.g., patient or HCP) before they are used to automatically program the medical device 402 (at 916). Depending on the implementation, the computed therapy settings (from 914) can be adjusted/changed algorithmically prior to being used to automatically program the medical device 402, or adjusted/changed by a user prior to being used to automatically program the medical device 402. For example, in one embodiment, a GUI can be displayed at the non-medical device 406 that includes an interface element that requests confirmation of the therapy settings by a user (e.g., the specific patient or a health care provider). This way, the user can confirm whether the therapy settings (from 914) are acceptable or whether they need to be changed/adjusted, in which case the user can change/adjust one or more of the therapy settings. When the user inputs information to indicate that the computed therapy settings (from 914) are not acceptable and should not be used to program the medical device 402, the user can input one or more adjusted therapy settings, and the method 900 then proceeds to 916. When the user inputs information to confirm that the therapy settings are acceptable, therapy settings can be used at 916 without being adjusted or changed. In another embodiment, an algorithm that can be implemented at the non-medical device 406 can confirm whether the computed therapy settings (from 914) are to be used in programming therapy settings (at 916). The algorithm can employ various artificial intelligence and/or machine learning technologies to determine whether the computed therapy settings (from 914) are appropriate for use as the programming therapy settings (at 916), and if not, can adjust/change one or more value(s) of the computed therapy settings (from 914) to other value(s) that are acceptable for use in programming therapy settings (at 916).

At 916, a processor device or controller of the medical device 402 can be automatically programmed (e.g., without user input at medical device 402) with the set of therapy settings for the specific patient to complete set up of the medical device 402 so that the medical device is properly configured for the specific patient. The set of therapy settings for the specific patient that are used to automatically program the medical device 402 can be either the set of therapy settings that were computed at 914, or a set of adjusted therapy settings that were adjusted/changed after being computed at 914.

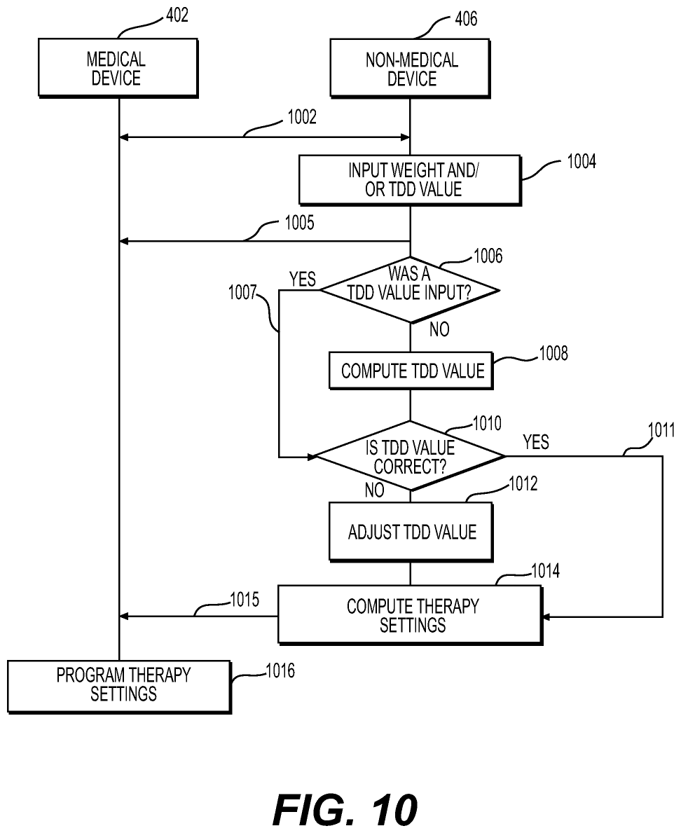

FIG. 10 is a flow diagram that illustrates a method 1000 for configuring therapy settings of a medical device 402 for a specific patient during startup mode of the medical device 402 in accordance with the disclosed embodiments.

In some embodiments, the method 1000 starts at 1002, where a non-medical device 406 establishes a communication link with the medical device 402. Alternatively, in another embodiment, the medical device 402 can establish a communication link with the non-medical device 406. In either of these embodiments, the communication link can be established automatically or in response to action by the specific patient that causes these communication links to be established.

At 1004, patient data can be input via the non-medical device 406 (e.g., a device of the patient or a health care provider). In some embodiments, the patient data can be input using a graphical user interface of the non-medical device 406 (e.g., during a pairing process with the medical device 402). In another embodiment, the patient data can be entered via a health care provider via another non-medical device. The patient data can include one or more of the weight of the specific patient and/or a value (Units) of a TDD of insulin for the specific patient. As described above with reference to FIG. 8, in some embodiments, the patient data that is input at 904 can also include other product and/or patient health information.

At 1005, the patient data can optionally be sent from the non-medical device 406 to the medical device 402. At 1006, the non-medical device 406 can determine whether a value of the TDD was input as part of the patient data. When the non-medical device 406 determines that a value of the TDD was input (as indicated by arrow 1007) as part of the patient data (at 1004), the method 1000 can proceed to 1010.

When the non-medical device 406 determines that a value of the TDD was not input as part of the patient data (at 1004), the method 1000 can proceed to 1008, where the non-medical device 406 can compute a value of the TDD using the weight of the patient (that was input at 1004). The computation at 1008 can be performed as described above with reference to FIG. 8.

At 1010, the non-medical device 406 can confirm whether the value of the TDD, which can be a value input by a user (e.g., patient or HCP) at 1004 or a value that was computed as a function of the patient's weight at 1008, should be used in computing therapy settings (at 1014), or changed/adjusted (at 1012) either algorithmically or by a user.

In some embodiments, at 1010, a GUI can be displayed at the non-medical device 406 that includes an interface element that requests confirmation of the value by a user (e.g., the specific patient or a health care provider). This way, the user can confirm whether the computed value of the TDD (from 1004 or 1008) is acceptable and should be used to compute therapy settings (at 1014) or whether the computed value of the TDD should be adjusted/changed at 1012. A user of the non-medical device 406 can confirm the value of the TDD (at 1010) or adjust the value of the TDD (at 1012).

When the user inputs information (at 1010) to confirm that the value of the TDD (from 1004 or 1008) is acceptable and should be used to compute therapy settings, the value of the TDD can be sent (at 1011) to the medical device 402. By contrast, when the user inputs information (at 1010) to indicate that the value of the TDD (from 1004 or 1008) is not acceptable and should not be used to compute therapy settings, the method proceeds to 1012, where the value of the TDD can be adjusted/changed. Depending on the embodiment, the value of the TDD can be adjusted/changed either based on a user input or algorithmically. For instance, in one embodiment, at 1012, the user (e.g., patient or HCP) can input an adjusted value of the TDD that should be used to compute therapy settings. In another embodiment, at 1012, an algorithm at the non-medical device 406 can adjust/change the value of the TDD to an adjusted value of the TDD that should be used to compute therapy settings. In some embodiments, the algorithm can employ various artificial intelligence and/or machine learning technologies to determine whether the value of the TDD is appropriate for use in computing the therapy settings, and if not, the algorithm can adjust value of the TDD to a value that is acceptable for use in computing the therapy settings.

At 1014, the non-medical device 406 can compute therapy settings locally based on the value of the TDD, which can be a value input by a user (e.g., patient or HCP) at 1004, a value that was computed at 1008 or an adjusted value that was adjusted/changed at 1012. The set of therapy settings for the specific patient that are computed at 1014 can include, but are not limited to, for example: a basal rate for the specific patient, an insulin sensitivity factor for the specific patient, an insulin to carbohydrate ratio for the specific patient, an active insulin time for the specific patient, a maximum bolus limit for the specific patient, a maximum basal rate for the specific patient, a bolus increment for the specific patient, a basal increment for the specific patient, etc. In one non-limiting implementation, the therapy settings can be computed as described above with reference to FIG. 8.

Although not illustrated in FIG. 10, in one embodiment, after the therapy settings are computed at 1014, the therapy settings can be displayed at the non-medical device 406 for confirmation or adjustment by the user (e.g., patient or HCP) before they are used to automatically program the medical device 402 (at 1016). Depending on the implementation, the computed therapy settings (from 1014) can be adjusted/changed algorithmically prior to being used to automatically program the medical device 402, or adjusted/changed by a user prior to being used to automatically program the medical device 402 (e.g., without user input at medical device 402). For example, in one embodiment, a GUI can be displayed at the non-medical device 406 that includes an interface element that requests confirmation of the therapy settings by a user (e.g., the specific patient or a health care provider). This way, the user can confirm whether the therapy settings (from 1014) are acceptable or whether they need to be changed/adjusted, in which case the user can change/adjust one or more of the therapy settings. When the user inputs information to indicate that the computed therapy settings (from 1014) are not acceptable and should not be used to program the medical device 402, the user can input one or more adjusted therapy settings, and the method 1000 then proceeds to 1016. When the user inputs information to confirm that the therapy settings are acceptable, therapy settings can be used at 1016 without being adjusted or changed. In another embodiment, an algorithm that can be implemented at the non-medical device 406 can confirm whether the computed therapy settings (from 1014) are to be used in programming therapy settings (at 1016). The algorithm can employ various artificial intelligence and/or machine learning technologies to determine whether the computed therapy settings (from 1014) are appropriate for use as the programming therapy settings (at 1016), and if not, can adjust/change one or more value(s) of the computed therapy settings (from 1014) to other value(s) that are acceptable for use in programming therapy settings (at 1016).

At 1015, the non-medical device 406 can send the computed therapy settings (or alternatively the adjusted therapy settings) to the medical device 402. In addition, as described above with reference to FIG. 8, in some embodiments, product and/or patient health information can also be communicated to the medical device 402 at 1015 (or alternatively at 1005).

At 1016, a processor device or controller of the medical device 402 can be automatically programmed with the set of therapy settings for the specific patient to complete set up of the medical device 402 so that the medical device is properly configured for the specific patient. The set of therapy settings for the specific patient that are used to automatically program the medical device 402 can be either the set of therapy settings that were computed at 1014, or a set of adjusted therapy settings that were adjusted/changed after being computed at 1014.

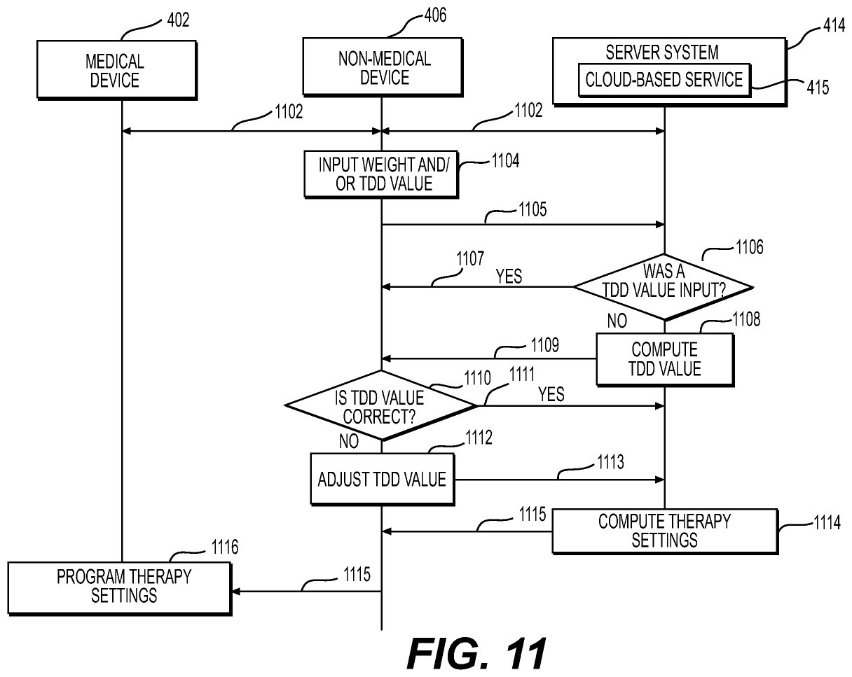

FIG. 11 is a flow diagram that illustrates another method 1100 for configuring therapy settings of a medical device 402 for a specific patient during startup mode of the medical device 402 in accordance with the disclosed embodiments. In some embodiments, the method 1100 starts at 1102, where a non-medical device 406 establishes a communication link with the medical device 402 and the server system 414 that hosts the cloud-based service 415. Alternatively, in another embodiment, the medical device 402 can establish a communication link with the non-medical device 406 and the server system 414. In another embodiment, the server system 414 can establish a communication link with the medical device 402 and the non-medical device 406. In any of these embodiments, the communication link can be established automatically or in response to action by the specific patient that causes these communication links to be established.

At 1104, patient data can be input via the non-medical device 406 (e.g., a device of the patient or a health care provider). In some embodiments, the patient data can be input using a graphical user interface of the non-medical device 406 (e.g., during a pairing process with the medical device 402). In another embodiment, the patient data can be entered via a health care provider via another non-medical device. The patient data can include one or more of the weight of the specific patient and/or a value (Units) of a TDD of insulin for the specific patient. As described above with reference to FIG. 8, in some embodiments, the patient data that is input at 904 can also include other product and/or patient health information.

At 1105, the patient data can be sent, for example, from the non-medical device 406 to the cloud-based service 415 at the server system 414. At 1106, the cloud-based service 415 can determine whether a value of the TDD was input as part of the patient data. When the cloud-based service 415 determines (at 1106) that a value of the TDD was input as part of the patient data (at 1104), the method 1100 can proceed to 1110.

When the cloud-based service 415 determines (at 1106) that a value of the TDD was not input as part of the patient data (at 1104), the method 1100 can proceed to 1108, where the cloud-based service 415 computes a value of the TDD using the weight of the patient that was input at 1104. The computation at 1108 can be performed as described above with reference to FIG. 8.

The value of the TDD that is computed (at 1108) can be sent (at 1109) by the cloud-based service 415 to the non-medical device 406 for review or confirmation. At 1110, the non-medical device 406 can determine whether the value of the TDD, which can be a value input by a user (e.g., patient or HCP) at 1104 or a value that was computed as a function of the patient's weight at 1108, should be used in computing therapy settings (at 1114), or changed/adjusted (at 1112) either algorithmically or by a user.

In some embodiments, at 1110, a GUI can be displayed at the non-medical device 406 that includes an interface element that requests confirmation of the value by a user (e.g., the specific patient or their health care provider). This way, the user can confirm whether the computed value of the TDD (from 1104 or 1108) is acceptable and should be used to compute therapy settings (at 1114) or whether the computed value of the TDD should be adjusted/changed at 1112. A user of the non-medical device 406 can confirm the value of the TDD (at 1110) or adjust the value of the TDD (at 1112).

When the user inputs information (at 1110) to confirm that the value of the TDD (from 1104 or 1108) is acceptable and should be used to compute therapy settings, the value of the TDD can be sent (at 1111) to the cloud-based service 415. By contrast, when the user inputs information (at 1110) to indicate that the value of the TDD (from 1104 or 1108) is not acceptable and should not be used to compute therapy settings, the method proceeds to 1112, where the value of the TDD can be adjusted/changed. Depending on the embodiment, the value of the TDD can be adjusted/changed either based on a user input or algorithmically. For instance, in one embodiment, at 1112, the user (e.g., patient or HCP) can input an adjusted value of the TDD that should be used to compute therapy settings. In another embodiment, at 1112, an algorithm at the non-medical device 406 can adjust/change the value of the TDD to an adjusted value of the TDD that should be used to compute therapy settings. In some embodiments, the algorithm can employ various artificial intelligence and/or machine learning technologies to determine whether the value of the TDD is appropriate for use in computing the therapy settings, and if not, the algorithm can adjust value of the TDD to a value that is acceptable for use in computing the therapy settings.

At 1114, the cloud-based service 415 can compute therapy settings based on the value of the TDD, which can be a value input by a user (e.g., patient or HCP) at 1104, a value that was computed at 1108 or an adjusted value that was adjusted/changed at 1112. In this embodiment, the cloud-based service 415 can compute the therapy settings, communicate the therapy settings to the non-medical device 406, and the non-medical device 406 can communicate the therapy setting to the medical device 402 (at 1115). In another embodiment, the cloud-based service 415 can compute the therapy settings, and communicate the therapy settings directly to the medical device 1102. The set of therapy settings for the specific patient that are computed at 1114 can include, but are not limited to, for example: a basal rate for the specific patient, an insulin sensitivity factor for the specific patient, an insulin to carbohydrate ratio for the specific patient, an active insulin time for the specific patient, a maximum bolus limit for the specific patient, a maximum basal rate for the specific patient, a bolus increment for the specific patient, a basal increment for the specific patient, etc. In one non-limiting implementation, the therapy settings can be computed as described above with reference to FIG. 11.

Although not illustrated in FIG. 11, in one embodiment, after the therapy settings are computed at 1114 and sent to the non-medical device 406 (at 1115), the non-medical device 406 can confirm or adjust/change one of more of the therapy settings before they are sent to and used to automatically program the medical device 402 (at 1116). The computed therapy settings (from 1114) can be changed algorithmically or adjusted by a user prior to being sent to and used to automatically program the medical device 402 (e.g., without user input at medical device 402).

For example, in one embodiment, a GUI can be displayed at the non-medical device 406 that includes an interface element that requests confirmation of the therapy settings by a user (e.g., the specific patient or a health care provider). This way, the user can confirm whether the therapy settings (from 1114) are acceptable or whether they need to be changed/adjusted, in which case the user can change/adjust one or more of the therapy settings. When the user inputs information to indicate that the computed therapy settings (from 1114) are not acceptable and should not be used to program the medical device 402, the user can input one or more adjusted therapy settings, and the method 1100 then proceeds to 1116. When the user inputs information to confirm that the therapy settings are acceptable, therapy settings can be used at 1116.

In another embodiment, the computed therapy settings (from 1114) can be confirmed for use in programming therapy settings (at 1116) via an algorithm that can be implemented at the non-medical device 406. The algorithm can employ various artificial intelligence and/or machine learning technologies to determine whether the computed therapy settings (from 1114) are appropriate for programming therapy settings (at 1116), and if not, can adjust one or more value(s) of the computed therapy settings (from 1114) to other value(s) that are acceptable for use in programming therapy settings (at 1116).

At 1115, the cloud-based service 415 can compute the therapy settings, communicate the therapy settings to the non-medical device 406, and the non-medical device 406 can communicate the computed therapy settings (or alternatively the adjusted therapy settings) to the medical device 402. In addition, as described above with reference to FIG. 8, in some embodiments, product and/or patient health information can also be communicated to the medical device 402 at 1115.

At 1116, a processor device or controller of the medical device 402 can be automatically programmed with the set of therapy settings for the specific patient to complete set up of the medical device 402 so that the medical device is properly configured for the specific patient. The set of therapy settings for the specific patient that are used to automatically program the medical device 402 can be either the set of therapy settings that were computed at 1114, or a set of adjusted therapy settings that were adjusted/changed after being computed at 1114.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be configurable to be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements

While at least one exemplary embodiment has been presented in the foregoing detailed description, it should be appreciated that a vast number of variations exist. For example, although the detailed description describes specific examples where the medical device being configured is an insulin infusion or delivery device for the treatment of diabetes, it should be appreciated that the same or similar principles can be used to configure other types of medical devices for embodiments addressing different medical conditions by changing the types of input data entered by the physician, the therapy types and parameters, etc. It should also be appreciated that the exemplary embodiment or embodiments described herein are not intended to limit the scope, applicability, or configuration of the claimed subject matter in any way. Rather, the foregoing detailed description will provide those skilled in the art with a convenient road map for implementing the described embodiment or embodiments. It should be understood that various changes can be made in the function and arrangement of elements without departing from the scope defined by the claims, which includes known equivalents and foreseeable equivalents at the time of filing this patent application.

What is claimed is:

1. A method comprising:
obtaining, at a computing system, prescription information for a patient, wherein the prescription information comprises a device prescription;
transforming, by the computing system, the prescription information into first configuration data comprising therapy settings for a first medical device referenced by the device prescription; and
causing, by the computing system, automatic configuration of the first medical device based on communicating the first configuration data to the first medical device.

2. The method according to claim 1, wherein the device prescription comprises:
identifying information for the patient, a model of the first medical device, and a therapy type prescribed for the patient.

3. The method according to claim 1, wherein the prescription information further comprises one or more of:
basic patient-specific data for the patient, the basic patient-specific data comprising:
physical, metabolic or anatomical data about the patient, and information about medical conditions;
patient lifestyle data for the patient, the patient lifestyle data comprising: data regarding a diet of the patient, and data regarding exercise characteristics of the patient;
information about a medical condition being targeted for therapy and information about medical conditions other than the condition being targeted for therapy;
information regarding therapy history of the patient; and
known therapy settings from prior medical devices of the patient.

4. The method according to claim 1, wherein transforming the prescription information into the first configuration data, comprises:
selecting an appropriate therapy configuration for the first medical device based on the prescription information; and
determining therapy parameters and settings for the first medical device based on the prescription information.

5. The method according to claim 4, wherein the appropriate therapy configuration comprises:
one or more therapy algorithms to be executed by the first medical device for the patient; and
one or more operating modes of the first medical device to be used for the patient.

6. The method according to claim 4, wherein the therapy parameters and settings comprise:
appropriate device configuration parameters or settings for configuring the first medical device of the patient; and
therapy configuration parameters for configuring the first medical device of the patient.

7. The method according to claim 4, further comprising:
combining, by the computing system, the appropriate therapy configuration and the therapy parameters and settings for the first medical device into the first configuration data; and
maintaining the first configuration data in a database.

8. The method according to claim 1, wherein the first configuration data is structured as at least one software image.

9. The method of claim 1, further comprising:
transforming, by the computing system, the prescription information into second configuration data comprising therapy settings for a second medical device referenced by the prescription information, wherein the first medical device is a different model from the second medical device.

10. The method of claim 1, wherein the first medical device comprises at least one of a group including an insulin infusion device and a glucose sensor.

11. A computing system, comprising:
at least one processor device; and
a non-transitory processor-readable medium operatively associated with the at least one processor device, the processor-readable medium comprising executable instructions configurable to cause the at least one processor device to perform a method comprising:
obtaining prescription information for a patient, wherein the prescription information comprises a device prescription;
transforming the prescription information into first configuration data comprising therapy settings for a first medical device referenced by the device prescription; and
causing automatic configuration of the first medical device based on communicating the configuration data to the first medical device.

12. The computing system of claim 11, wherein the device prescription comprises:
identifying information for the patient, a model of the first medical device, and a therapy type prescribed for the patient.

13. The computing system of claim 11, wherein the prescription information comprises one or more of:
basic patient-specific data for the patient, the basic patient-specific data comprising: physical, metabolic or anatomical data about the patient, and information about medical conditions;
patient lifestyle data for the patient, the patient lifestyle data comprising: data regarding a diet of the patient, and data regarding exercise characteristics of the patient;
information about a medical condition being targeted for therapy and information about medical conditions other than the condition being targeted for therapy;
information regarding therapy history of the patient; and
known therapy settings from prior medical devices of the patient.

14. The computing system of claim 11, wherein transforming the prescription information into the first configuration data comprises:
selecting an appropriate therapy configuration for the first medical device based on the prescription information; and
determining therapy parameters and settings for the first medical device based on the prescription information.

15. The computing system of claim 14, wherein the appropriate therapy configuration comprises:
one or more therapy algorithms to be executed by the first medical device for the patient; and
one or more operating modes of the first medical device to be used for the patient.

16. The computing system of claim 14, wherein the therapy parameters and settings comprise:
appropriate device configuration parameters or settings for configuring the first medical device of the patient; and
therapy configuration parameters for configuring the first medical device of the patient.

17. The computing system of claim 14, wherein the computing system further comprises storage, and wherein the method further comprises:
combining the appropriate therapy configuration and the therapy parameters and settings for the first medical device into the first configuration data; and
maintaining the first configuration data in a database.

18. The computing system of claim 11, wherein the first configuration data is structured as at least one software image.

19. The computing system of claim 11, wherein the method further comprises:
transforming the prescription information into second configuration data comprising therapy settings for a second medical device referenced by the prescription information, wherein the first medical device is a different model from the second medical device.

20. The computing system of claim 11, wherein the first medical device comprises at least one of a group including an insulin infusion device and a glucose sensor.
